(19) United States
(12) Patent Application Publication
(10) Pub. No.: US 2026/0055459 A1
(43) Pub. Date: Feb. 26, 2026

(54) SIMPLIFIED ANALYSIS METHOD OF DNA AND CELL FREE DNA AND USES THEREOF

(71) Applicant: YUNNAN DAORAN SCIENCE AND TECHNOLOGY LTD., Kunming, Yunnan (CN)

(72) Inventors: Donald Arthur Leigh, Kunming (CN); David Stephen Cram, Kunming (CN); Li Wang, Kunming (CN)

(21) Appl. No.: 19/105,446

(22) PCT Filed: Mar. 22, 2023
(86) PCT No.: PCT/CN2023/082988

§ 371 Date: Feb. 21, 2025

(30) Foreign Application Priority Data
Aug. 22, 2022 (WO) PCT/CN2022/113833

(51) Int. Cl. C12Q 1/6879; C12N 15/10; C12Q 1/6806; C12Q 1/6869

(52) U.S. Cl. CPC C12Q 1/6879; C12N 15/1065; C12Q 1/6806; C12Q 1/6869

(57) ABSTRACT

Provided are an amplification-free method for the preimplantation genetic testing (PGT) of in-vitro fertilization (IVF) embryos, an amplification-free method of generating a library for the PGT, and a method of identifying the genetic background of IVF embryos. Provided are also a method of determining the degree of non-embryonic DNA contamination, and an amplification-free method of generating a library of cell-free DNAs (cfDNAs) from a biological material. Similarly provided are a method for enrichment of methylated fragments from the amplification free library, and a method for identifying methylation profiles and/or changes of same, in samples of different sources.

### TECHNICAL FIELD

The present invention relates to techniques for DNA genetic testing. In particular, the present invention relates to an amplification-free method for the simplified analysis of low amounts of DNA and cell free DNA such as found in the preimplantation genetic testing (PGT) of in-vitro fertilization (IVF) embryos, micro-biopsies and human body fluids.

### BACKGROUND

In the natural process of cell growth, minute amounts of DNA can be released into the cellular environment. This DNA is typically in a degraded form of 10s to 1000s of base pairs. All cells have a natural life span, at the end of which cell degradation processes will reduce chromosomes to fragments typically of several hundred base pairs and multimers representative of nucleosome structures-often referred to as apoptosis. When cellular tissues suffer from injury, internal processes can instigate a chromosome degradation process, again breaking down the DNA into smaller fragments but in a less ordered way to yield less defined fragments. Any or all of these processes can occur in cells that are in culture, such as tissue culture or embryo culture or in complex living systems, such as a human body.

Culture systems are closed environments, typically in a culture dish with cells surrounded by nutrient rich medium. It is in this medium that any nucleic acids released from the cell system will accumulate and/or be slowly further degraded. Having been released from the original cultured cells, the DNA is likely to be representative of the genetics of the remaining living cells. The total amount of this degraded DNA is typically very small-too small to make any direct measurements of the amount. Since it is often not always possible nor appropriate to take a large amount of the cultured cells to analyse the genetics, some sort of amplification process is typically considered. Most of the current, common processes of amplification, often termed whole genome amplification, will in reality fail to amplify some types of DNA present and may over-amplify others, introducing imbalances into any subsequent chromosome profile. It is possible to semi correct such imbalances using advanced bioinformatics processes but this will not always be successful and may lead to incorrect interpretations regarding cell genetics. In addition some important secondary information that may be important for some interpretations, such as fragment length and any DNA modifications present in the fragments will be lost during the amplification process. A method or an approach that enables analysis of small amounts of DNA without altering the profile or losing the secondary information may be advantageous for workers wanting detailed information about the cultured cell system.

Natural complex cellular systems, such as in a human, have many different cell types each with common general chromosome profile but each also with their own life spans and DNA modification profiles. They are part of a multicellular system and are interconnected by the circulatory system which both feeds them and also removes waste products including extra cellular DNA (cfDNA) associated with the natural growth and death processes. The circulating blood therefor carries the many different DNA signatures of each of the different cell types that are interconnected. Similarly, the excretory systems such as urine, can also carry some of these degraded DNA fragments with the associated signatures. While the body contains a large number of cells that are undergoing the death process and release of degraded DNA at any one time, continuous clearance of the DNA occurs in the liver as well as excretion via the bladder and urinary system means that such amounts of DNA remain relatively small. Generally, the chromosome and genetic profile of this DNA will be common throughout the body but each cell type and each organ can have unique patterns of secondary DNA modifications such as that can be used to identify their contribution to the overall cell free DNA profile in the plasma component of the blood or in the urine. Changes in relative amounts of cell free DNA or individual organ contribution can be used to indicate a disease status or cell damage brought about by injury or infection. Typically, a large volume of plasma is used as a source of cell free DNA which can then be purified and quantified. A method that can simply quantify the relative amount of cfDNA in the cell free plasma, may be useful to help identify any underlying tissue damage or infection that might be present. The ability to identify which organ(s) is/are releasing the DNA can assist in the diagnosis of the potential injury site. Such injury may be the result of physical or chemical processes or may be associated with underlying disease processes such as cancer or metabolic imbalances such as diabetes, other chronic disease conditions or simply the ageing process. Currently, the most common approach to identify which organ or tissue is the source of the cfDNA, is to purify a relatively large amount of DNA and then chemically modify it so that the underlying DNA signatures can be analysed in some manner, such as DNA sequencing. This process requires a large amount of DNA starting material, much of which is destroyed during the DNA treatment process. In addition, the subsequent analysis of this modified/treated DNA is complex and requires special bioinformatics for interpretation. The possibility of a simple process that requires less starting DNA, fewer handling steps, less interventions and reduces the necessity for complex and specialized bioinformatics may be of benefit in routine applications such as identification and health management of chronic diseases, cancer investigations, non-invasive prenatal investigations, organ of origin studies and many other investigations.

The genome is a complex composite of different DNA pieces-many of which may not be of immediate interest for analysis purposes. High complexity may mean less efficient use of resources, such as DNA sequencing, when only a limited target range is under review. There are several methods available to reduce this complexity to make subsequent analysis more efficient and cost effective. Such procedures may involve the directed amplification of specific target points contained within the DNA complex (for example PCR-polymerase Chain reaction, LAMP-Loop Mediated Isothermal Amplification, MLPA-Multiplex ligation-dependent Probe Amplification as well as systems based on bacterial and virus recombination and repair such as CRISPR)—these however require detailed knowledge of each target site, primer design and development and usually require a DNA target that is intact for the region of interest. In addition, some elements of the original target information arc lost (e.g., fragment start/end points as well as internal modifications of DNA bases) due to the amplification approach. In addition, accessing more than one target in a single reaction can also be limited by the amplification procedure used or may require extensive development work to ensure compatibility between each specific probe/target combination. While some DNA, such as chromosome DNA from tissue and cell preparations, are ostensibly intact for most targets, other preparations, such as cfDNA may be significantly damaged which can preclude amplification of some or even all the wanted targets by the standard amplification methods-especially when DNA starting amounts are very small. A method that enables selective DNA enrichment from the starting source and is less sensitive to the status of the DNA in terms of its degradation profile while also not requiring extensive pre-workup for multiple targets would assist in the analysis of many different DNA sources for genetic profiling. If such a method also maintained original information, including original fragment features such as start and end points or secondary aspects such as DNA base modifications, it may prove advantageous in many different applications. A method that could also maintain the identification of original target strands would be useful to remove any bias in target representation if an amplification process was necessary for any subsequent analysis. An alternative to directed amplification, is the capture of wanted regions of DNA by a homologous probe that utilizes specific DNA-base pairing interactions. Such systems are commercially available and may be liquid/solution based (such as Agilent or IDT probes) or liquid/solid based (such as arrays from Agilent or other suppliers). These systems typically require large amounts of DNA as input—a potential problem if only limited starting DNA is available. A method that enables otherwise hidden but wanted features of the DNA to simultaneously be preserved while also permitting reduction of the complexity of the DNA source by enrichment could be useful in many areas, including cancer analysis, prenatal analysis and chronic disease assessment. Examples for specific application areas include and are not limited to embryo assessment for improving assisted reproduction technique, cancer detection and treatment monitoring, chromosome profiles, determining allelic imbalances, and cell based DNA analysis.

Similar problems for analyzing cfDNA or very small amounts of genomic DNA also exist in other application areas. Accordingly, instead of the current approaches, the simplified analysis method of the present disclosure can be equally applicable and beneficial to analyze samples from many different biological sources.

### SUMMARY OF THE INVENTION

The present disclosure provides an approach for preparing limited amounts of DNA for sequencing libraries suitable for next generation sequencing or for selective enrichments of desired fragments.

In a first aspect, the present disclosure provides an amplification-free method for the analyzing a biological sample comprising
i) providing a sample containing cell-free DNA (cfDNA);
ii) repairing the cfDNA fraction to obtain single base overhang fragments;
iii) adding a directional barcoded sequencing compatible adapter to each of the ends of the repaired cfDNA, thereby generating a barcoded library for sequencing; and
iv) sequencing the barcoded library.

In a second aspect, the present disclosure provides an amplification-free method of generating a library for the preimplantation genetic testing (PGT) of in-vitro fertilization (IVF) embryos comprising
i) providing a sample containing cell-free DNA (cfDNA) from the spent medium in the culture of an IVF embryo;
ii) repairing the cfDNA to obtain single base overhang fragments; and
iii) adding a directional barcoded adapter to each of the ends of the repaired cfDNA, thereby generating a barcoded library of the spent media cfDNA for sequencing.

In a third aspect, the present disclosure provides a method of identifying the genetic background of in-vitro fertilization (IVF) embryos comprising
i) providing a sample containing cell-free DNA (cfDNA) from the spent medium in the culture of an IVF embryo;
ii) repairing the cfDNA to obtain single base overhang fragments; and
iii) adding a directional barcoded adapter to each of the ends of the repaired cfDNA, thereby generating a barcoded library; and
iv) determining a 24 chromosome copy number profile, a genome-wide heterozygous SNP profile or mitochondrial sequence by sequencing the barcoded library.

In a fourth aspect, the present disclosure provides an amplification-free method of determining the degree of non-embryonic DNA contamination in the spent medium in the culture of an IVF embryo, comprising
i) providing a sample containing cell-free DNA (cfDNA) from the spent medium in the culture of an IVF embryo;
ii) repairing the cfDNA to obtain single base overhang fragments; and
iii) adding a directional barcoded adapter to each of the ends of the repaired cfDNA, thereby generating a barcoded library for sequencing;
iv) sequencing the library; and
v) calculating the mitochondrial DNA (mtDNA) to chromosome ratio, wherein a higher mtDNA to chromosome ratio is indicative of a lower degree of non-embryonic DNA contamination, and a lower mtDNA to chromosome ratio is indicative of a higher degree of non-embryonic DNA contamination.

In a fifth aspect, the present disclosure provides an amplification-free method of generating a library of cell-free DNAs (cfDNAs) from a biological material, the method comprising
i) providing a sample containing cell-free DNA (cfDNA) from the biological material;
ii) repairing the cfDNA to obtain single base overhang fragments; and
iii) adding a directional barcoded adapter to each of the ends of the repaired cfDNA, thereby generating a barcoded library for sequencing.

In the sixth aspect of the invention, there is provided an amplification-free method for preparing the sample for methylation profile analysis, the method comprising
i) providing a sample containing cell-free DNA (cfDNA) from the biological material;
ii) repairing the cfDNA to obtain single base overhang fragments; and
iii) adding a directional barcoded adapter to each of the ends of the repaired cfDNA, thereby generating a barcoded library and avoiding PCR amplification which destroys the methylation patterns by converting methyl-C to C.

In the seventh aspect of the present disclosure, there is provided an amplification-free method for examining a selected genome region on a chromosome associated with a gene, the method comprising:
i) providing a sample containing cell-free DNA (cfDNA)
ii) repairing the cfDNA fraction to obtain single base overhang fragments;
iii) adding a directional barcoded sequencing-compatible adapter to each of the ends of the repaired cfDNA, thereby generating a barcoded library;
iv) enriching the library for the selected genome region; and
v) sequencing the barcoded library.

In the eighth aspect of the present disclosure, there is provided an amplification-free method for examining a selected genome region on a chromosome associated with a polymorphic site, the method comprising:
i) providing a sample containing cell-free DNA (cfDNA)
ii) repairing the cfDNA fraction to obtain single base overhang fragments;
iii) adding a directional barcoded sequencing-compatible adapter to each of the ends of the repaired cfDNA, thereby generating a barcoded library;
iv) enriching the library for the selected genome region; and
v) sequencing the barcoded library.

### BRIEF DESCRIPTION OF THE DRAWINGS

FIG. 1 shows a schematic of how the Simplified Analysis Method can be used for direct analysis or can be further processed to gain more specific information about the (cf) DNA sample.

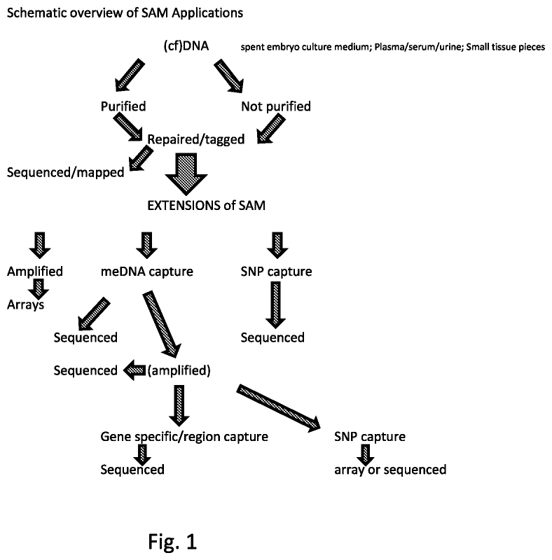

FIG. 2 shows the comparison of the amplification-free method for generating libraries of the present disclosure to WGA based method for the analysis of culture media.

FIG. 3 shows the size of the native cfDNA in embryo spent media, in which panels A, B, C and D represent four separate samples.

FIG. 4 shows the analysis of different DNA size fractions, <200 bp (A) and >200 bp pairs (B) for the reads corresponding to Chromosome 21, Chromosome 22 and Chromosome X.

FIG. 5 shows the determination of sex chromosome balance, in particular, the depth of reads mapped to autosomes and Chromosome X in a 46, XY sample (A) and a 46, XX sample (B), and the mapping of Chromosome Y specific sequences in two 46, XY samples and two 46, XX samples (C).

FIG. 6 shows the ploidy of embryos, including a 46, XY euploid embryo (A), a 46, XX euploid embryo (B) and an aneuploid embryo lacking a copy of Chromosome 15 (C).

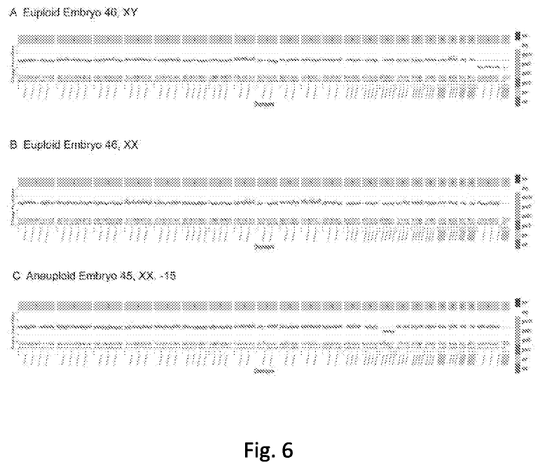

FIG. 7 shows the possible sources of non-embryonic DNA contamination.

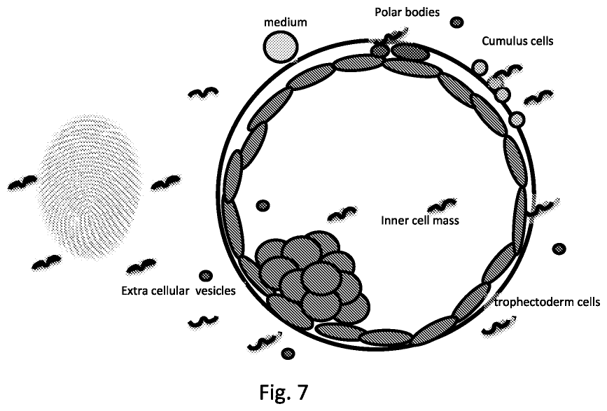

FIG. 8 shows the analysis of mitochondrial DNA.

FIG. 9 shows sequencing methylation plots from various cfDNA and tissue sources. Depicted are changes in methylation capture across genomic sites.

FIG. 10 Gene probe capture plots of various (cf) DNA sources. Depicted are changes in meDNA capture at different genes signifying change in methylation status for the gene

FIG. 11. SNP probe ratios from a model system depicting different minor allele amounts (4%-8%).

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., molecular genetics, bioinformatics, developmental biology, and IVF).

Unless otherwise indicated, the techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al., (editors) Current Protocols in Immunology, John Wiley & Sons (including all updates until present).

As used herein, the term about, unless stated to the contrary, refers to +/−10%, more preferably +/−5%, of the designated value.

As used herein, the word “comprise” or variations thereof will be understood to imply the inclusion of a stated element, integer or step, or group of elements, integers or steps, but not the exclusion of any other element, integer or step, or group of elements, integers or steps.

As used herein, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

As used herein, the term “screening” refers to a process of assessing an embryo. Such a process can be used to select a suitable embryo for transferring into a female's uterus, for example. In a similar manner, the term “screening” may refer to assessing any DNA sample to determine if it is at risk of having an ancuploid genome, a pathogenic genetic variation or a polymorphism of interest to the investigator.

As used herein, the term “cuploidy” or “cuploid” means a cell, an embryo or a DNA sample that comprises one or more complete genomes (two for human) without any redundant chromosome, and the term “aneuploidy” or “aneuploid” means a cell, an embryo or a DNA sample comprises at least one incomplete genome or at least one redundant chromosome or a part thereof.

The term “pathogenic” is used herein in reference to genetic variations that are known to be or predicted to be linked to a disease. The association of one or more genetic variations with a disease can result in the disease or can represent the genetic predisposition, i.e. risk, of developing the discasc.

As used herein, the terms “aligned”, “alignment”, or “aligning” refer to one or more sequences that are identified as a match in terms of the order of their nucleic acid molecules to a known sequence from a reference genome. Such alignment can be done manually or by a computer algorithm, examples including the Efficient Local Alignment of Nucleotide Data (ELAND) computer program distributed as part of the Illumina Genomics Analysis pipeline. The matching of a sequence read in aligning can be a 100% sequence match or less than 100% (non-perfect match).

The term “allele” herein refers to a sequence variant of a genetic sequence. For purposes of this application, alleles can but need not be located within a gene sequence. Alleles can be identified with respect to one or more polymorphic positions such as SNPs, while the rest of the gene sequence can remain unspecified. For example, an allele may be defined by the nucleotide present at a single SNP, or by the nucleotides present at a plurality of SNPs.

The term “sequencing” herein refers to a method for determining the nucleotide sequence of a polynucleotide, e.g. genomic DNA. Preferably, sequencing methods include as non-limiting examples NGS methods (i.e., high throughput sequencing methods), NGS in which clonally amplified DNA templates or single DNA molecules are sequenced in a massively parallel fashion (Volkerding et al., 2009; Metzker et al., 2010).

The term “sequencing read” or “read” refers to a DNA sequence of sufficient length (e.g., at least about 30 bp) that can be used to identify a larger sequence or region, e.g. that can be aligned and specifically assigned to a chromosome or genomic region or gene.

The term “whole genome amplification” herein refers to a process whereby genomic DNA sequences present in a sample are amplified to provide multiple copies of the genome that the sequences represent.

The term “haplotype” refers to a DNA sequence comprising one or more genetic variation of interest contained on a subregion of a single chromosome of an individual. The genetic variations of a haplotype can be of the same type, e.g. all SNPs, or can be a combination of two or more types of genetic variations, e.g. combinations of SNPs and STRs. A haplotype can refer to a set of genetic variations in a single gene, an intergenic sequence, or in larger sequences include both gene and intergenic sequences, e.g., a collection of genes, or of genes and intergenic sequences. For example, a haplotype can refer to a set of genetic variations in the regulation of complement activation (RCA) locus, which includes gene sequences for complement factor H (CFH), FHR3, FHR1, FHR4, FHR2, FHR5, and F13B and intergenic sequences (i.e., intervening intergenic sequences, upstream sequences, and downstream sequences that are in linkage disequilibrium with genetic variations in the genic region). A haplotype, for instance, can be a set of maternally inherited alleles, or a set of paternally inherited alleles, at any locus.

The term “haplotyping” herein refers to a process for determining one or more haplotypes in an individual and includes use of family pedigrees, molecular techniques and/or statistical inference. Preferably, haplotypes are determined by sequencing using next generation sequencing technologies.

The term “adapter” herein refers to a compatible nucleotide fragment that can be ligated or otherwise attached to the fragments of repaired DNA. Such an adapter may be double stranded DNA or DNA analogue or RNA or RNA analogue and has a defined sequence compatible with any subsequent process such as DNA sequencing or DNA amplification. A Y adapter has a single stranded and a double stranded region that enables it to be directionally added to the fragments of interest.

### In Vitro Fertilisation (IVF)

The methods described herein can be used to screen IVF embryos, such that embryos which are at risk of having an aneuploid genome or a pathogenic genetic variation can be identified, thereby permitting suitable embryos to be selected for transfer. IVF is a process of fertilisation where an egg is combined with sperm outside the body, i.e., in vitro. The process involves monitoring and in some instances stimulating a female's ovulatory process, removing an ovum or ova (egg or eggs) from the female's ovaries and letting sperm fertilise them in a suitable liquid in a laboratory setting. After the fertilised egg (zygote) undergoes embryonic culture for about 2-7 days, it is implanted in the same or another female's (e.g., a surrogate's) uterus, with the intention of establishing a successful pregnancy. Typically, parents will undergo IVF if they either have difficulty conceiving naturally, or are at risk of transmitting a genetic disease to the embryo.

The methods described herein are suitable for IVF embryos of any animal, provided that a suitable reference genome is available so that sequencing data can be aligned to identify potential genetic variations. For example, the embryo may be a human or other non-human animal embryo. In some embodiments, the embryo is a human embryo. In other embodiments, the embryo is a bovine, ovine, equine, porcine, canine, feline, or other non-human animal embryo.

Obtaining sequencing data for an “embryo”, as described herein, includes sequencing cfDNA from spent medium of an embryo fertilized not less than about 40 hours before sampling, including a blastocyst (typically an embryo at day 4, day 5, day 6 or day 7 after fertilization). Thus, in some embodiments, the embryo is a two-day, three-day, four-day, five-day, six-day, or seven-day old embryo. The plural form of this term is included, such that, the term “an embryo” as used herein contemplates that more than one embryo or blastocyst may be concurrently screened or transferred according to the methods of the present disclosure.

“Transferring” an IVF embryo refers to the process of placing an IVF embryo into a female subject, with the objective that the embryo will implant and result in a viable pregnancy. The female subject may be the female parent of the embryo or any other suitable female for transfer of the embryo, for example in the case of a surrogate pregnancy.

As contemplated herein, the methods of the present disclosure may be used to screen one or more embryos concurrently such that more than one IVF embryo deemed not at risk of having an aneuploid genome or a pathogenic genetic variation may be identified and transferred. The number of such embryos that may be appropriate to transfer may be determined by one of skill in the art according to conventional methods.

The terms “genetic variation”, “polymorphism” and “variant” are used interchangeably herein to refer to the occurrence of a variation in the genetic sequence of the embryo's genome (or the genome of either parent) relative to a reference genome.

Similarly, any DNA sample can be screened concurrently. The number of samples screened may be determined by one of skill in the art according to conventional methods.

The terms “genetic variation”, “polymorphism” and “variant” are used interchangeably herein to refer to the occurrence of a variation in the genetic sequence of the DNA sample relative to a reference genomc.

Genetic variations encompass sequence differences that include single nucleotide polymorphisms (SNPs), tandem SNPs, small-scale multi-base deletions or insertions, called “indels” (also called deletion insertion polymorphisms or DIPs), Multi-Nucleotide Polymorphisms (MNPs), Short Tandem Repeats (STRs), restriction fragment length polymorphism (RFLP), deletions, including microdeletions, insertions, including microinsertions, duplications, inversions, translocations, multiplications, complex multi-site variants, copy number variations (CNV), and other structural variations comprising any other change of sequence in a chromosome.

The term “single nucleotide polymorphism (SNP)” refers to a single base (nucleotide) polymorphism in a DNA sequence among individuals in a population.

An “indel” is an insertion or deletion of bases in the genome of an organism. It is typically classified among small genetic variations, measuring from 1 to 10 000 base pairs in length.

As used herein, the term “structural variation” is used to refer to any variation in structure of an organism's chromosome. It comprises many kinds of variation in the genome, and usually includes microscopic and submicroscopic types, deletions, duplications, copy-number variants, insertions, inversions and translocations. Typically, structural variations affect a larger portion of sequence than SNPs but smaller than a chromosome abnormality (though the definitions have some overlap).

The term “copy number variation” herein refers to a type of structural variation that is a variation in the number of copies of a nucleic acid sequence that is typically about 1 kb or larger present in a test sample in comparison with the copy number of the nucleic acid sequence present in a qualified sample.

### Examples for Specific Application Areas of the Invention

### 1. Embryo Assessment for Improving Assisted Reproduction Technique

It has been observed that for any couple presenting for IVF, a significant portion of the embryos generated during the IVF process do not have a balanced set of chromosomes. The transfer of such embryos will commonly result in failure to initiate the pregnancy, but for those embryos that do start a pregnancy, miscarriage or abnormal fetal development, or in some cases, a live birth with a syndromic phenotype. The proportion of chromosomally unbalanced embryos will generally be greater with increasing maternal age but even young women may have some or even all of their embryos with unbalanced chromosome sets. Therefore, all women undergoing IVF may derive some benefit from embryo euploid identification and selection. In addition, patients with a genetic predisposition can also benefit from both euploid and disease-free embryo selection for transfer.

Currently, for PGT cycles performed worldwide, an invasive biopsy procedure is used to obtain one or several embryonic cells to test for chromosomes and/or an underlying genetic condition. Invasive biopsy with whole genome amplification (WGA) and analysis using arrays or next generation sequencing methods has become the standard to make the final embryo diagnosis. While the procedure has been well developed and is in widespread application in many PGT laboratories, there is concern in some circles that significant harm is being done to embryos with this invasive approach. These secondary embryo manipulations appear to have variable outcomes according to the clinic expertise, and the harm done to the embryo may outweigh any benefits of PGT. Additionally, a number of countries have legislation that prohibits for religious and/or philosophical reasons, any manipulation of embryos such as biopsy.

Therefore a non-invasive technology that permits testing of the embryonic DNA without the invasive steps could significantly benefit all women undertaking IVF in identifying euploid embryos for transfer. Such an approach could also allow other couples at genetic risk to have the option of non-invasive processes for embryo selection.

Recently, a small amount of cell-free DNA (cfDNA) of low molecular weight from both chromosomal and mitochondrial sources has been reported in the embryo spent culture media and in the fluid of the blastocoele cavity. Using paired-end sequencing of the cfDNA released from the blastocoele fluid of expanded embryos, it was shown that the predominant size of the cfDNA was about 156 bp. Other analyses focusing on the source of the cfDNA in embryo spent media, have identified significant contamination in up to 60% of samples from different sources including cumulus cells, first polar bodies, second polar bodies, sperm as well as from a principle component of most embryo media, the human serum albumin (HISA). In addition, the presence of any cells or micro-cells that typically contain DNA would also contribute to this background DNA by releasing their DNA with the use of any sample processing step involving heat or extraction processes.

All current non-invasive methods investigating this cfDNA in spent embryo media employ a PCR based WGA step to amplify the cfDNA followed by next generation sequencing or array copy number analysis to interpret the chromosome ploidy status of the embryo. All current WGA approaches use heat as a primary denaturing step and cycled heat protocols for the enzymatic amplification. This heating step will not only disrupt the target cfDNA molecules but will also disrupt any extraneous cells in the sample, including cells that may be linked to the embryo, the embryo associated structures or operator introduced cellular contamination. These cell-based contaminations may result in non-embryo DNA amplifying in preference to the wanted embryo target DNA molecules and potentially confounding or even swamping any subsequent embryo DNA based genomic analysis.

One of the physical limitations of most WGA methods is a size restriction on the target DNA that can be appropriately amplified. The lower limit for amplified DNA fragment size is generally greater than 200 bp. The WGA method is therefore intrinsically biased to target and amplify the larger fragments present in the spent culture media, thereby introducing a bias at the earliest stages of analysis and potentially making amplified libraries not representative of the original cfDNA, and hence, the embryonic genome. A further limitation of WGA, being based on a PCR-amplification process, is an inherent amplification bias with over and under representation of specific genomic sequences in the final amplified DNA preparation. With the intrinsic bias of WGA for larger targets, any large fragments of non-embryonic DNA contamination present in or introduced into the spent culture media will potentially be preferentially amplified to produce DNA libraries from both original embryonic cfDNA as well as any contaminating and introduced sources of both non-embryonic cfDNA and cellular based DNA.

WGA based approaches are relatively costly as well as time consuming taking up to three hours to generate DNA libraries with many steps requiring operator handling followed by additional procedures needed to purify and quantify the amplified DNA as well as to measure the quality of the libraries for sequencing. Further, after WGA, it is not possible to estimate the original size of any cfDNA that was present in the medium nor identify how much was originally present in the spent medium sample. It is not surprising therefore that to date, many non-invasive PGT-A results, published using different WGA assays, have high amplification failure rates, are variable, often contain discordances of ploidy compared to invasive trophectoderm results, are variable in the quality of the chromosome profiles and often yield no interpretable results. In many cases, WGA completely fails to generate DNA libraries suitable for sequencing analysis.

As an alternative to current invasive and non-invasive approaches for PGT, there is a need for a non-invasive technology where sample preparation is simple, rapid, reliable and accurate.

Accordingly, the present disclosure overcomes or at least alleviate some of the problems of prior art and to improve PGT outcomes for patients using a non-invasive approach for embryo testing.

### 2. Cancer Detection and Treatment Monitoring

Methylation is a key epigenetic DNA modification that regulates the fundamental cellular processes of transcription and gene expression. Studies have shown that alteration of methylation patterns through hypo- or hyper-methylation can lead to altered gene expression and the initiation and propagation of a variety of human disease conditions. Accordingly, changes in DNA methylation patterns may be indicative of underlying changes in organs and tissues and be used as a test for early disease detection or assessment.

The cfDNA circulating in the blood plasma fraction is fragmented DNA with a dominant fragment size of 166-176 base pairs. This cfDNA represents the genome breakdown products of tissue cells from many different parts of the body. Approximately 1-3% of the chromosomal DNA is methylated and so a similar fraction of the cfDNA will be similarly methylated. This methylated cfDNA, seen in plasma, reflects the general methylation status of normal somatic cells from different organs connected through the circulatory system. The different organs will each produce their own methylated DNA sub-signature sharing many of the same methylation modifications with other cell lineages but also displaying tissue-of-origin specific patterns, in some chromosome regions, that are unique or specific to that cell lineage. Changes in methylation patterns will reflect changes in tissue gene expression and hence mDNA profiles. Possible alterations in normal or typical cellular functions can therefore be analysed by changes in the usual methylation profile for that cell type. In cancer for example, normal cell functions are disrupted with uncontrolled cell growth and often with the gaining of invasive potential being associated with concomitant changes in specific DNA methylation sites. DNA methylation patterns of the tissue tumour are usually different to the original tissues, at least in some regions of the chromosome and these altered methylation signatures can subsequently appear in the plasma, serum or urine cfDNA fractions.

There are a variety of methods to detect DNA methylation including: gene-specific assays that use methylation specific modifications to effectively enable specific targeting by PCR; genome-wide assays that utilise chemical DNA modifications (bisulphite sequencing) and methylation specific arrays. More recently allele-specific DNA sequencing on 3rd generation nanopore based DNA sequencing platforms was shown to be able to detect and map methylation sites across the genome. In general though, the genome-wide assays developed to date are cumbersome, laborious and time consuming and are subject to many different errors. This results in the current methods being technically complex and not being cost-effective for routine applications in the analysis of methylated cfDNA.

As an alternative to current methylation detection approaches, there is a need for a technology that is simple, rapid, reliable and accurate. Thus, for early disease detection and monitoring the effectiveness of treatment, a simple non-invasive test that targets the methylated cfDNA fraction would be a significant advance in disease detection and monitoring.

Accordingly, it is an aspect of the present disclosure to overcome or at least alleviate some of the problems of prior art and to simplify genome-wide methylation detection as a means of assessing an individual's general health and well-being with respect to early signs of cancer or other chronic diseases.

### 3. Chromosome Profiles

Many cancer cells have altered regions of chromosomes, observed as losses and/or gains of whole chromosomes or regions of chromosomes. These changes in chromosome profile were traditionally observed using classical cytogenetic karyotyping but in recent years the more common platform(s) used for chromosome profile assessment is next generation sequencing (CNVseq) or array-based hybridization systems. These array systems can be simple sequence tag site and/or single nucleotide polymorphism probes. During early stages of cancer growth, there tends to be an evolving and constantly changing chromosome profile set amongst different sub clones of the tumors and their metastases. Often this reflects in a combination of apoptosis as well as necrosis of the masses—with each of these death processes having their own fragment profile. Any change in tissue growth dynamics can be reflected in changes in cfDNA-both the amount of DNA as well as the fragment profile and chromosome profile. Any method that enables a simple viewing of these features could assist in detection and course of underlying cancers and potentially facilitate the monitoring of treatments.

The placenta of a pregnant woman releases fragmented cfDNA of the fetus into the maternal circulatory system—this has become the basis for modern noninvasive prenatal screening (NIPS). The majority of cfDNA is of maternal origin but 4-20% is of fetal origin—while this is relatively little fetal DNA in a large maternal background, it is sufficient to identify major chromosome imbalances of fetal origin. Typically, the cfDNA from about one millilitre of blood is purified to yield ˜20 ng of cfDNA (this amount of DNA is the equivalent of 3,000 diploid cells and represents >10¹⁰ 160-200 bp fragments). This purified cfDNA is then used to make a library for array or next generation sequencing (NGS) based analysis. Array analysis however, may require more DNA necessitating an amplification step. NGS analysis needs only a smaller DNA amount but requires library preparation which typically involves some intermediate PCR type amplification process. Bioinformatics is used to reduce the possible biases of the chromosome profile(s) associated with amplification and multiple analysis of any original library fragments. Of the final library though, only a minute fraction is used for the final sequence analysis involving only ˜3×10⁶-8×10⁶ fragments.

Any technology that can reduce the number of intermediate steps and requires less cfDNA as a start can facilitate the handling and processing of NIPS samples. Similarly, avoiding the possibility of analysis of daughter strands will simplify the bioinformatics and at the same time, improve efficiency of the sequencing resource.

### 4. Determining Allelic Imbalances

As with chromosome profiles, alterations of allele ratios of any SNPs present are similarly indicators of the altered chromosome and/or chromosome segments often associated with developing cancers. Current approaches that involve a directed PCR amplification will be both limited in scope and also fail to account for many of the SNP targets due to the degradation profile of individual SNP targets.

A placenta in a pregnant woman will release cfDNA representative of the fetus—this will be both the maternal SNP contribution as well as the new paternal half set. Where these paternal alleles differ to the maternal alleles, a minor allele can be observed. A major to minor allele ratio can therefor be used to assess fetal cfDNA fraction in the predominant maternal cfDNA background. Similarly to chromosome profiles, disturbance within fetal cfDNA SNP ratio profiles across the different chromosomes may be used as an indicator of unbalanced chromosome ratios in the developing fetus.

An organ transplant patient will receive tissue that has a different allele combination in many regions of the different chromosomes. As the organ becomes accepted by the recipient's system, cells within the tissue of the transplanted organ will begin to undergo the same life cycle cell growth/death as the original organ. This life cycle will introduce any unique SNPs present in the donor tissue into the cfDNA of the recipient. Assessment of these SNPs as a ratio to the recipient SNP can be a useful indicator of whether the transplant is adapting to the new location or is failing. An elevated ratio after an initial settling in period may indicate organ rejection while an initial depressed ratio may indicate initial transplant failure.

A simple method that can assist in both the estimation of these allele ratios from cfDNA samples and indicate total relative cfDNA levels may be useful for monitoring the development of cancers or assessing treatment progression, assessment of fetal genome complement in a pregnancy or be used to monitor tissue/organ transplant successes.

### 5. Cell Based DNA Analysis

Often the amount of sample available for genomic screening is more than sufficient for using most current technology platforms. Occasionally though, the amount of tissue available for DNA analysis is limited. These may be fine needle biopsies from suspected tumors, slide mounted tissue sections, embryo biopsies or small tissue biopsies. The DNA from these can be extracted in the usual manner but are then typically subjected to some sort of whole genome amplification process-thus similarly losing some of the original information or introducing initial targeting biases the same as for cfDNA amplification. An alternative approach of fragmentation of the DNA can be performed with any of the currently available methods such as sonication or nuclease digestion. Whether it is amplified DNA or native DNA, sequence libraries can be prepared after fragmenting, using the simplified analysis method described herein. If elements of secondary information are not required in the analysis being performed, then the sequencing library can be amplified and utilized for procedures needing more DNA (such as arrays) or be a source material for enrichment procedures (such as SNP or selected gene targets). This amplification may introduce some level of bias in the final DNA profile but it is less likely to be the combinatorial biases of the initial amplification target initiation plus DNA sequence amplification variability. A method that can identify original fragments that were present at library construction commencement may be useful in analysing the original DNA fragments present. Similarly, method that can reduce the variable imbalances created by current PCR-type whole genome amplifications can be potentially useful in aiding both simple and more complex cell and tissue studies.

### Generation of Libraries

The present disclosure provides an amplification-free method of generating a library of cell-free DNAs (cfDNAs) from a biological material, the method comprising
i) providing a sample containing cell-free DNA (cfDNA) from the biological material;
ii) repairing the cfDNA and preferably obtaining single base overhang fragments; and
iii) adding a barcoded adapter to each of the ends of the repaired cfDNA, thereby generating a barcoded library for sequencing.

Similarly, the method can be carried out with DNA derived from various cell samples, not limited to the spent medium in the culture of an IVF embryo. Therefore, the present disclosure provides an amplification-free method of generating a library for the preimplantation genetic testing (PGT) of in-vitro fertilization (IVF) embryos comprising
i) providing a sample containing cell-free DNA (cfDNA) from the spent medium in the culture of an IVF embryo;
ii) repairing the cfDNA and preferably obtaining single base overhang fragments; and
iii) adding a directional barcoded adapter to each of the ends of the repaired cfDNA, thereby generating a barcoded library for sequencing.

The present disclosure provides a library generated by the method of the invention.

The methods for obtaining DNAs, such as cfDNA from spent medium in a biological sample such as the culture of an IVF embryo are known in the art.

More conveniently, the spent medium can be used directly without an isolation step. For example, embryos can be grown to the blastocyst stage in single media drops to allow genetic analysis of individual embryos. In some embodiments, the sample in step i) is the spent medium in the culture of an IVF embryo.

The cfDNA may comprise 5′- and/or 3′-overhanging ends, or internal nicks. Therefore, in some embodiments, repairing the cfDNA comprises converting 5′ and/or 3′ overhanging ends into blunt ends, and/or repairing the internal nicks.

Typically, the repairing can be achieved using methods or kits known in the art. The repaired fragments can be phosphorylated by enzymatic treatment, for example using polynucleotide kinase. In some embodiments, a single deoxynucleotide, e.g. deoxyadenosine (A), is added to the 3′-ends of the fragments, for example, by the activity of certain types of DNA polymerase such as Taq polymerase or Klenow exo minus polymerase. In some embodiments, the method further comprises a step of adding a 3′ dA overhang to each end of the repaired fragments.

dA-tailed products are compatible with ‘T’ overhang present on the 3′ terminus of each duplex region of adapters to which they are ligated in a subsequent step. dA-tailing prevents self-ligation of both of the blunt-ended polynucleotides such that there is a bias towards formation of the adapter-ligated sequences. The dA-tailed fragments are ligated to double-stranded adapter polynucleotide sequences. The same adapter can be used for both ends of the fragment, or two sets of adapters can be utilized. Ligation methods are known in the art and utilize ligase enzymes such as DNA ligase to covalently link the adapter to the d-A-tailed polynucleotide. The adapter may contain a 5′-phosphate moiety to facilitate ligation to the target 3′-OH. The dA-tailed fragment contains a 5′-phosphate moiety, either residual from the shearing process, or added using an enzymatic treatment step, and has been end repaired, and optionally extended by an overhanging base or bases, to give a 3′-OH suitable for ligation.

In some embodiments of the method of the present disclosure, the directional barcoded adapter is an Y adapter. In some embodiments, the Y adapter comprises a barcode in the double-stranded region. In some embodiments, the Y adapter comprises a barcode in the single-stranded region.

The products of the ligation reaction can be purified to remove unligated adapters, and or adapters that may have ligated to one another. Purification of the ligation products can be obtained by methods including gel electrophoresis and solid-phase reversible immobilization (SPRI). Purification can also remove enzymes, buffers, salts and the like to provide favorable reaction conditions for the subsequent step. In some embodiments, the method of the present disclosure comprises a step of purification.

Libraries with various barcodes can be sequenced in combination. Therefore, it is possible to prepare a combination of libraries. It should be noted that the purification may result in loss of cfDNA of interest, which is disadvantageous for subsequent steps. As the DNA level in the combination is significantly higher than a single library, and thus, the loss of DNA during the purification will be less disadvantageous. In some embodiments, the method comprises generating a plurality of barcoded libraries with different barcodes. In some embodiments, the method comprises a step of combining the plurality of barcoded libraries prior to the purification.

The cfDNAs may be bound to proteins, which will influence the sequencing. In some embodiments, the method comprises a step of removing proteins bound to the cfDNA. In some embodiments, the cfDNA is treated with a protease and/or detergents to remove nucleosome or heterochromatin structures from the cfDNA fragments.

As discussed previously, cfDNA is derived from spent medium of an embryo fertilized not less than about 40 hours before sampling, including a blastocyst (typically an embryo at day 4, day 5, day 6 or day 7 after fertilization. In some embodiments, the spent medium is collected from a blastocyst culture of Day 5 to Day 7.

In some embodiments, the sample comprises cfDNA extracted from cells or tissues. In some embodiments, the cfDNA is from tissue removed from an embryo by biopsy. The cfDNA can be further processed in a manner making it compatible with the subsequent library preparations.

### Screening of IVF Embryos

The inventors surprisingly found that the libraries generated by the above established method can be directly sequenced for the screening of IVF embryos suitable for implantation.

The present disclosure provides an amplification-free method for the preimplantation genetic testing (PGT) of in-vitro fertilization (IVF) embryos comprising
i) providing a sample containing cell-free DNA (cfDNA) from spent medium in the culture of an IVF embryo;
ii) repairing the cfDNA fraction and preferably obtaining single base overhang fragments;
iii) adding a directional barcoded adapter to each of the ends of the repaired cfDNA, thereby generating a barcoded library for sequencing; and
iv) sequencing the barcoded library.

In some embodiments, repairing the cfDNA comprises converting 5′ and or 3′ overhang into blunt ends, and/or repairing the internal nicks.

In some embodiments, the single base overhang fragments comprise a 3′ dA overhang to each end of the repaired fragments. In some embodiments, the Y adapter comprises a barcode in the double-stranded region. In some embodiments, the Y adapter comprises a barcode in the single-stranded region.

In some embodiments, the method of the present disclosure comprises a step of purification.

In some embodiments, the method comprises generating a plurality of barcoded libraries with different barcodes. In some embodiments, the barcoded adapters may be further modified to include a random nucleotide sequence that gives a unique sequence to that adapter. In some embodiments, the method comprises a step of combining the plurality of barcoded libraries prior to the purification.

In some embodiments, the method comprises a step of removing proteins bound to the cfDNA. In some embodiments, the cfDNA is treated with a protease and/or detergent to remove nucleosome or heterochromatin structures from the cfDNA fragments.

In some embodiments, the spent medium is collected from a blastocyst culture of Day 5 to Day 7.

Any nucleic acid sequencing platform is suitable for performing sequencing of the genomic DNA, including high-throughput DNA sequencing methods (also commonly referred to as “next-generation sequencing” or “NGS”). Thus, in some embodiments, the barcoded library is sequenced using a high throughput sequencing method. In some embodiments, the library is sequenced using DNA nanoball sequencing. In some embodiments, the DNA nanoball sequencing is performed with combinatorial probe anchor ligation (cPAL). The sequencing can be a paired-end sequencing or an unpaired-end sequencing, preferably a paired-end sequencing.

NGS methods provide sequence reads that vary in size from tens to hundreds of base pairs. In some embodiments of the method described herein, the sequence reads are about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. It is expected that technological advances will enable single-end reads of greater than 500 bp, enabling for reads of greater than about 1000 bp when paired end reads are generated. In one embodiment, the sequence reads are 36 bp. Other sequencing methods that can be employed by the method of the invention include the single molecule sequencing methods that can sequence nucleic acids molecules >5000 bp. The massive quantity of sequence output is transferred by an analysis pipeline that transforms primary imaging output from the sequencer into strings of bases. A package of integrated algorithms performs the core primary data transformation steps: image analysis, intensity scoring, base calling and alignment.

In some embodiments, the sequencing data covers at least 10,000 mapped sequencing reads or at least 0.02% of the human genome.

The method may comprise one or more additional steps for analyzing the data resulting from the sequencing. In some embodiments, the method further comprises the analysis of the sequencing data to obtain a chromosome profile, such as a 24-chromosome profile for human, for determining chromosome ploidy status of an embryo. In some embodiments, the method further comprises the analysis of the sequencing data to obtain a chromosome X and Y profile, e.g., of a human embryo, for determining sex chromosome balances. In some embodiments, the method further comprises calculating the copy number of X chromosome by comparison to autosomal regions.

For assessing sex chromosome balance, the software has been designed to first calculate the X chromosome copy number by comparison to autosomal regions. This identifies whether, relative to the autosomes, there is one copy of chromosome X or two copies present. For confirmation against a possible background of low level male DNA in the HISA component of the culture media, Y-specific gene sequences or other Y-unique sequences from multi-copy genes such as TSPY (17-30 copies) or the heterochromatin are then analysed for relative amounts. 46,XX embryos will reveal the background of Y-specific sequences from HISA male DNA contamination. Thus, designated 46,XY embryos are confirmed by a higher level of Y-specific sequences than female embryos.

The sequencing data can be analyzed by aligning to a reference genome so that any differences between the embryo's genome sequence (and the parents) and the reference can be identified as potential genetic variations. In some embodiments, the reference genome is a human reference genome. In some embodiments, the reference genome is a Genome Reference Consortium Human Build. In some embodiments, the reference genome is Genome Reference Consortium Human Build 37 (GRCh37) or Genome Reference Consortium Human Build 38 (GRCh38) or any future build (i.e., Build 39 or later builds).

The cfDNA size profiles can be used to bioinformatically separate or enrich the embryonic DNA fraction relative to non-embryonic DNA fraction. Thus, an embryonic or an enriched embryonic fraction of the sequencing data can be selected for analysis, improving the reliability and accuracy of the embryo genetic diagnosis. This is analogous to non-invasive prenatal diagnosis where it is known that the fetal cfDNA fragments in maternal plasma are generally smaller in size than the maternal cfDNA fragments which can then assist in determining the fetal fraction of the overall cfDNA. Therefore, in some embodiments, the method comprises a step of fractioning the reads based on the size profile.

The present disclosure also provides a method of identifying the genetic background of in-vitro fertilization (IVF) embryos comprising
i) providing a sample containing cell-free DNA (cfDNA) from the spent medium in the culture of an IVF embryo;
ii) repairing the cfDNA preferably obtaining single base overhang fragments; and
iii) adding a directional barcoded adapter to each of the ends of the repaired cfDNA, thereby generating a barcoded library; and
iv) determining the genome-wide heterozygous SNP profile or mitochondrial sequence by sequencing the barcoded library.

In some embodiments, repairing the cfDNA comprises converting 5′ and or 3′ overhang into blunt ends, and/or repairing the internal nicks.

In some embodiments, the single base overhang fragments comprise a 3′ dA overhang to each end of the repaired fragments. In some embodiments, the Y adapter comprises a barcode in the double-stranded region. In some embodiments, the barcoded adapters may be further modified to include a random nucleotide sequence that gives a unique sequence to that adapter. In some embodiments, the Y adapter comprises a barcode in the single-stranded region.

In some embodiments, the method of the present disclosure comprises a step of purification. Standard techniques for nucleic acid isolation and purification are known and are described in, for example, in Miller (ed.) 1972 Experiments in Molecular Genetics, Cold Spring Harbor Laboratory, Cold Spring Harbor, N.Y.; Old and Primrose, 1994 Principles of Gene Manipulation, 5th ed., University of California Press, Berkeley; Schleif and Wensink, 1982 Practical Methods in Molecular Biology; Glover (Ed.) 1985 DNA Cloning: Vols. I AND II, IRL Press, Oxford, UK; Harnes and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York City.

In some embodiments, the method comprises generating a plurality of barcoded libraries with different barcodes. In some embodiments, the barcoded adapters may be further modified to include a random nucleotide sequence that gives a unique sequence to that adapter. In some embodiments, the method comprises a step of combining the plurality of barcoded libraries prior to the purification.

In some embodiments, the method comprises a step of removing proteins bound to the cfDNA. In some embodiments, the cfDNA is treated with a protease to remove nucleosome or heterochromatin structures from the cfDNA fragments.

In some embodiments, the spent medium is collected from a blastocyst culture of Day 5 to Day 7.

In some embodiments, the method comprises haplotyping to determine the genome-wide heterozygous SNP profile.

The sequencing data can be analyzed by aligning to a reference genome so that any differences between the embryo's genome sequence (and the parents) and the reference can be identified as potential genetic variations. In some embodiments, the reference genome is a human reference genome. In some embodiments, the reference genome is a Genome Reference Consortium Human Build. In some embodiments, the reference genome is Genome Reference Consortium Human Build 37 (GRCh37) or Genome Reference Consortium Human Build 38 (GRCh38) or any future build (i.e., Build 39 or later builds). The reference genome includes the nuclear genome and mitochondrial genome, such as the mitochondrial genome of the female parent.

The genetic background includes genetic variations, especially pathogenic variations, such as single nucleotide polymorphisms (SNPs), insertions or deletions (indels), copy number variations (CNVs), and/or structural variations.

For single gene analysis, the software can be designed to search for heterozygous single nucleotide polymorphisms (SNPs) for linkage analysis and genetic disease prediction.

For SNP analysis and potentially for diagnosis of mitochondrial disease, it may be needed to amplify the cfDNA in library of the invention, which is generated by an amplification-free method, to generate sufficient material for subsequent approaches such as further enrichment of selected DNA targets, arrays, sequencing or real-time PCR. Nucleic acid amplification methods are also well known, including polymerase chain reaction (PCR) (PCR Protocols, A Guide to Methods and Applications, ed. Innis, Academic Press, N.Y. 1990; PCR: A Practical Approach, M. J. McPherson, et al., IRL Press (1991)); ligase chain reaction (LCR) (Landegren et al., 1988); transcription amplification (Kwoh et al., 1989); self-sustained sequence replication (Guatelli et al., 1990); Q Beta replicase amplification (Smith et al., 1997), and other RNA polymerase mediated techniques such as nucleic acid sequence based amplification, NASBA (U.S. Pat. Nos. 4,683,195 and 4,683,202); 3SR (self-sustained sequence reaction); RACE-PCR (rapid amplification of cDNA ends); PLCR (a combination of polymerase chain reaction and ligase chain reaction); SDA (strand displacement amplification); and SOE-PCR (splice overlap extension PCR).

Pathogenic genetic variations can be identified, for example, by querying a database of known genetic variations which is annotated with their level of pathogenicity. Alternatively, for a genetic variation which is either not in such a database, or for which its level of pathogenicity is uncertain, pathogenicity prediction algorithms can be used to determine whether that variation is likely to be pathogenic.

Suitable databases of known pathogenic genetic variations include ClinVar (https://www.ncbi.nlm.nih.gov/clinvar/), CLINVITAE (http://clinvitae.invitae.com/), Leiden Open Variant Database (LOVD; http://www.lovd.nl/), Human Genetic Variation Database (HGVD; http://www.hgvd.genome.med.kyoto-u.ac.jp/), Online Mendelian Inheritance in Man (OMIM; https://www.omim.org/), EGL's Variant Classification Catalog (EmVClass; http://www.egl-curofins.com/cmvclass/cmvclass.php), ARUP mutation database (http://www.arup.utah.edu/database/), or Carver allele-specific variation database (https://www.carverlab.org/database).

A number of computer algorithms are available for aligning sequences, including, without limitation, BLAST, BLITZ, FASTA, BOWTIE, ELAND (Illumina, Inc., San Diego, Calif., USA), Burrows-Wheeler Aligner (Li and Durbin, 2010), or GATK (DePristo et al., 2011; Mckenna et al., 2010). Analysis of sequencing information for the identification of polymorphic sequences may allow for a small degree of mismatch (0-2 mismatches per sequence tag) to account for minor polymorphisms that may exist between the reference genome and the embryo or parent genomes.

In order to avoid misdiagnoses due to amplification errors such as allelic or locus drop out, it is understood herein that sequencing an allele of interest may include sequencing nucleic acid around the allele to ensure amplification accuracy. For example, the disease-causative allele may be physically linked (close together) with a non-causative allele nearby in the DNA sequence. These two sites in the DNA are very likely to be inherited together, barring any meiotic recombination between the sites. Sites nearer each other are less likely to undergo recombination. As a result, the non-causative allele can be used as a confirmatory marker of the disease causing allele in order to avoid misdiagnosis from disease allele PCR dropout. Such techniques are familiar to one of skill in the art and include “haplotyping”. Suitable methods include those described in WO 14145820 and WO 15051006.

In some embodiments, the sample comprises cfDNA extracted from cells or tissues. In some embodiments, the cfDNA is from tissue removed from an embryo by biopsy. The cfDNA can be further processed in a manner making it compatible with the subsequent library preparations.

### Determination of Non-Embryonic DNA in the Sample

It is known that the culture media may contain significant maternal DNA contamination relative to embryonic DNA. The media is subsequently used without any discreet or separate cfDNA purification step.

Therefore, the present disclosure further provides an amplification-free method of determining the degree of non-embryonic DNA contamination in the spent medium in the culture of an IVF embryo, comprising
i) providing a sample containing cell-free DNA (cfDNA) from the spent medium in the culture of an IVF embryo;
ii) repairing the cfDNA preferably obtaining single base overhang fragments; and
iii) adding a directional barcoded adapter to each of the ends of the repaired cfDNA, thereby generating a barcoded library for sequencing;
iv) sequencing the library; and
v) calculating the mtDNA to chromosome ratio,
wherein a higher mtDNA to chromosome ratio is indicative of a lower degree of non-embryonic DNA contamination, and a lower mtDNA to chromosome ratio is indicative of a higher degree of non-embryonic DNA contamination.

In some embodiments, repairing the cfDNA comprises converting 5′ and or 3′ overhang into blunt ends, and/or repairing the internal nicks.

In some embodiments, the single base overhang fragments comprise a 3′ dA overhang to each end of the repaired fragments. In some embodiments, the Y adapter comprises a barcode in the double-stranded region. In some embodiments, the barcoded adapters may be further modified to include a random nucleotide sequence that gives a unique sequence to that adapter. In some embodiments, the Y adapter comprises a barcode in the single-stranded region.

In some embodiments, the method of the present disclosure comprises a step of purification.

In some embodiments, the method comprises generating a plurality of barcoded libraries with different barcodes. In some embodiments, the barcoded adapters may be further modified to include a random nucleotide sequence that gives a unique sequence to that adapter. In some embodiments, the method comprises a step of combining the plurality of barcoded libraries prior to the purification.

In some embodiments, the method comprises a step of removing proteins bound to the cfDNA. In some embodiments, the cfDNA is treated with a protease to remove nucleosome or heterochromatin structures from the cfDNA fragments.

In some embodiments, the spent medium is collected from a blastocyst culture of Day 5 to Day 7.

In some embodiments, the mtDNA to chromosome ratio is mtDNA to autosome ratio.

The sequencing data can be analyzed by aligning to a reference genome so that any differences between the embryo's genome sequence (and the parents) and the reference can be identified as potential genetic variations. In some embodiments, the reference genome is a human reference genome. In some embodiments, the reference genome is a Genome Reference Consortium Human Build. In some embodiments, the reference genome is Genome Reference Consortium Human Build 37 (GRCh37) or Genome Reference Consortium Human Build 38 (GRCh38) or any future build (i.e., Build 39 or later builds). The reference genome includes the nuclear genome and mitochondrial genome, such as the mitochondrial genome of the female parent.

The level of maternal DNA contamination in a 46,XY embryo can be determined by assessing the X chromosome copy number. In some embodiments, the embryo is a human 46,XY embryo, and the method further comprises a step of assessing the X chromosome copy number. An increased copy number between 1 and 2 is indicative of maternal contamination. The absolute copy number value of X can therefore predict the % of maternal DNA contamination. In an extension of this analysis, if there is aneuploidy seen for a specific chromosome, the copy number should also be affected similarly to the X chromosome and show an intermediate copy number between 1 and 2 reflective of the non-embryonic X chromosome contamination.

In some embodiments, the sample comprises cfDNA extracted from cells or tissues. In some embodiments, the cfDNA is from tissue removed from an embryo by biopsy. The cfDNA can be further processed in a manner making it compatible with the subsequent library preparations.

### Methylation Profile Analysis

The present disclosure provides an amplification-free method for preparing the sample for methylation profile analysis, the method comprising
i) providing a sample containing cell-free DNA (cfDNA) from the biological material;
ii) repairing the cfDNA and preferably obtaining single base overhang fragments; and
iii) adding a directional barcoded adapter to each of the ends of the repaired cfDNA, thereby generating a barcoded library and avoiding PCR amplification which destroys the methylation patterns by converting methyl-C to C.

In a preferred embodiment, the adapter comprises unique Y adapters with incorporated barcoding. This enables directional addition of the adapters and also permits more samples to be sequenced per individual run. Since PCR duplication is avoided, more effective reads per sample are obtained thus significantly increasing information for data analysis. Alternative sequence primers/adapters can be utilised, allowing sequencing to be performed on different NGS platforms or 3rd generation sequencing platforms.

In a preferred embodiment, barcoded libraries from different samples can be combined into a single sample in preparation. Such a combination is cost effective for the enrichment of multiple methylated cfDNA fractions.

In a preferred embodiment, the method comprises paired end sequencing of the Y adaptor library fragments to provide the full-length sequence of each fragment. Such a sequencing improves the mapping of methylated fragments and maximises the methylation data available for analysis.

In a preferred embodiment, the Y adaptors can be modified to have a random sequence added to each primer strand in the single strand region thus giving each and every strand a unique, identifiable 5′ and 3′ section. Such a tagging would permit direct identification of all original unique targets containing specific methylation sites.

The generated library comprises both methylated and non-methylated cfDNA. In a preferred embodiment, the method comprises enriching the methylated fraction of the combined cfDNA libraries of the present disclosure, e.g., by specific capture of the methylated cfDNA fragments.

In a preferred embodiment, the specific capture is carried out using antibodies with the specificity for double or single stranded methylated DNA fragments, including anti-mC monoclonal or polyclonal antibodies or anti-ds antibodies from patients with SLE disease.

In a preferred embodiment, the antibodies such as anti-mC antibodies are bound to or captured by enrichment matrix such as beads to efficiently enrich the methylated fragments, allowing the non-methylated cfDNA to be washed away and the methylated cfDNA fraction to be eluted into a single tube for NGS, further prepared for nanopore sequencing or subjected to further enrichments such as selected gene or intergenic region capture. For example, the cfDNA library is enriched by hybridization to a gene chip or in solution with DNA probes designed from known methylated regions or using other matrices that preferentially bind methylated DNA (mDNA) which can then be differentially eluted and sequenced.

In a preferred embodiment, the bound methylated DNA fragments are released from the enrichment matrix and sequenced without further modifications to reveal a global methylation profile of the DNA fragment library. Such profiles can be generated by simple mapping and binning against standard chromosome references.

In a preferred embodiment, the method further comprises simply amplifying the enriched DNA fragments using primers homologous to the directional adapter sequences originally added prior to mDNA capture. Since the enrichment of methylated fragments has already occurred, there is no loss of information in subsequent amplification processes. Precise methylation signatures lost (only methylated regions) will be identified.

In a preferred embodiment, the method comprises improving DNA recovery with protcasc. The protease can digest any protein(s) bound to the DNA or other protein structures in the plasma. This step is designed to remove proteins, preferably all protein structures, (nucleosome and heterochromatin) bound to the DNA fragments, exposing hidden methylation sites and improving the efficiency of DNA library preparation and methylation detection.

In a preferred embodiment, the protease is known to be efficient at digesting and removing protein structures bound to DNA, such as that selected from the group consisting of trypsin and proteinase K.

In a preferred embodiment, the protease is a heat labile proteinase K or membrane bound trypsin. Such protease can be inactivated or removed thereby avoiding the interference with sample preparation, e.g., downstream molecular processing of the DNA including DNA repair, A addition and ligation.

In some embodiments, the methods further comprises sequencing the methylated DNA, e.g., by NGS.

In a preferred embodiment, the full DNA sequence of the methylated DNA fragments is obtained in NGS using the sequencing primers of the Y adaptors.

In a preferred embodiment, the NGS is performed in paired end NGS sequencing mode to derive the full-length sequence of each enriched methylated DNA fragment, maximising the sequencing information for methylated regions.

In a preferred embodiment, nanopore sequencing is used to obtain the full DNA sequence of long concatenated enriched methylated DNA fragments, in terms of the bases A, G, C, T and methylated C.

In a preferred embodiment, the method further comprises analysing the sequencing data derived from methylated DNA fragments with novel algorithms. In a preferred embodiment, the method comprises analysing the NGS data to map the methylated regions to chromosome bins and generate the methylation profiles for analysis. In a preferred embodiment, the method comprises analysing the nanopore sequencing data to plot the genome-wide methylation sites for methylation analysis.

In some embodiments, the method identifies genome regions of hypermethylation and hypomethylation. In some embodiments, the method identifies specific genomic sites of hypermethylation and hypomethylation.

The method can be used to predict a disease state (such as cancer and other chronic conditions) according to changes in parts of the genome methylation profile. The method can also be used to enrich for other selected regions of the genome such as polymorphic sites or known mutation sites. Such information can be used for assessing relative contributions of different tissues to a final cfDNA profile. Further, the method permits multiple different enrichment stages of cfDNA.

In some embodiments, the sample comprises cfDNA extracted from cells or tissues. In some embodiments, the cfDNA is from tissue removed from an embryo by biopsy. The cfDNA can be further processed in a manner making it compatible with the subsequent library preparations.

### Examining a Selected Genome Region

The present disclosure provides an amplification-free method for examining a selected genome region on a chromosome associated with a gene, the method comprising:
i) providing a sample containing cell-free DNA (cfDNA);
ii) repairing the cfDNA fraction and preferably obtaining single base overhang fragments;
iii) adding a directional barcoded sequencing-compatible adapter to each of the ends of the repaired cfDNA, thereby generating a barcoded library;
iv) enriching the library for the selected genome region; and
v) sequencing the barcoded library.

In a preferred embodiment, the library is enriched by selectively binding any cfDNA homologous to the selected genome region with hybridization capture probes. The selected genome region(s) may include known gene exonic or intronic sites or regions of the chromosome associated with mutations, methylation regions or otherwise of interest to the investigator.

Preferably, the sequence can be assessed for relative ratios of any polymorphic points identified in the captured DNA fragments. Such information being useful for assessing contributions of different tissues such as transplanted organs, spontancous chromosome changes associated with cancers, pregnancies or similar where distinguishable sequences can be beneficial in assessing genetic features of the tissue or aid in assessing the general health of the different tissues.

In a preferred embodiment, the cfDNA can be cell associated but has been extracted and processed into a form compatible with the library preparation and subsequent manipulations.

The present disclosure provides an amplification-free method for examining a selected genome region on a chromosome associated with a polymorphic site, the method comprising:
i) providing a sample containing cell-free DNA (cfDNA);
ii) repairing the cfDNA fraction and preferably obtaining single base overhang fragments;
iii) adding a directional barcoded sequencing-compatible adapter to each of the ends of the repaired cfDNA, thereby generating a barcoded library;
iv) enriching the library for the selected genome region; and
v) sequencing the barcoded library.

In a preferred embodiment, the library is enriched by selectively binding any cfDNA homologous to the selected genome region with hybridization capture probes. The selected genome region(s) may include polymorphic single nucleotide sites or regions of other polymorphic nature of interest to the investigator.

In a preferred embodiment, the selected genome region can be sequenced and analysed for variations such as single base substitutions or small indels.

Preferably, the sequence can be assessed for relative ratios of any polymorphic points identified in the captured DNA fragments. Such information being useful for assessing contributions of different tissues such as transplanted organs, spontaneous chromosome changes associated with cancers, pregnancies or similar where distinguishable sequences can be beneficial in assessing genetic features of the tissue or aid in assessing the general health of the different tissues.

In a preferred embodiment, the cfDNA can be cell associated but has been extracted and processed into a form compatible with the library preparation and subsequent manipulations.

In some embodiments, the sample comprises cfDNA extracted from cells or tissues. In some embodiments, the cfDNA is from tissue removed from an embryo by biopsy. The cfDNA can be further processed in a manner making it compatible with the subsequent library preparations.

### Embodiments

1. An amplification-free method for the preimplantation genetic testing (PGT) of in-vitro fertilization (IVF) embryos comprising
   i) providing a sample containing cell-free DNA (cfDNA) from spent medium in the culture of an IVF embryo;
   ii) repairing the cfDNA fraction and preferably obtaining single base overhang fragments;
   iii) adding a directional barcoded adapter to each of the ends of the repaired cfDNA, thereby generating a barcoded library for sequencing; and
   iv) sequencing the barcoded library, and optionally
   v) fractioning the reads based on the size profile.
2. The method of Embodiment 1, wherein repairing the cfDNA comprises converting 5′ and/or 3′ overhang into blunt ends, and/or repairing the internal nicks.
3. The method of Embodiment 1, wherein the single base overhang fragments comprise a 3′ dA overhang to each end of the repaired fragments.
4. The method of Embodiment 1, wherein the barcoded library is sequenced by paired end sequencing.
5. The method of Embodiment 1, wherein the barcoded adapter is an Y adapter.
6. The method of Embodiment 5, wherein the Y adapter comprises a barcode in the double-stranded region.
7. The method of embodiment 6, wherein the Y adapter contains a random nucleotide sequence of 1-10 bases in the single-stranded region of each arm 8. The method of any of Embodiments 1 to 7, further comprising a step of removing proteins bound to the cfDNA, e.g., the cfDNA is treated with a protease to remove nucleosome or heterochromatin structures from the cfDNA fragments.
9. The method of any of Embodiments 1 to 8, wherein the method comprises generating a plurality of barcoded libraries with different barcodes.
10. The method of any of Embodiments 1 to 9, further comprising a step of purification.
11. The method of Embodiment 10, comprising a step of combining the plurality of barcoded libraries prior to the purification.
12. The method of any of Embodiments 1 to 11, further comprising the analysis of the sequencing data to obtain a 24-chromosome profile for determining chromosome ploidy status of an embryo.
13. The method of any of Embodiments 1 to 11, further comprising the analysis of the sequencing data to obtain a chromosome X and Y profile for determining sex chromosome balances.
14. The method of Embodiment 13, further comprising calculating the copy number of X chromosome by comparison to autosomal regions.
15. The method of any of Embodiments 1-14, wherein the spent medium is collected from a blastocyst culture of Day 5 to Day 7.
16. An amplification-free method of generating a library for the preimplantation genetic testing (PGT) of in-vitro fertilization (IVF) embryos comprising
   i) providing a sample containing cell-free DNA (cfDNA) from the spent medium in the culture of an IVF embryo;
   ii) repairing the cfDNA and preferably obtaining single base overhang fragments; and
   iii) adding a directional barcoded adapter to each of the ends of the repaired cfDNA, thereby generating a barcoded library for sequencing.
17. The method of Embodiment 16, wherein repairing the cfDNA comprises converting 5′ and/or 3′ overhang into blunt ends, and/or repairing the internal nicks.
18. The method of Embodiment 16, wherein the single base overhang fragments comprise a 3′ dA overhang to each end of the repaired fragments.
19. The method of Embodiment 16, wherein the barcoded adapter is an Y adapter.
20. The method of Embodiment 19, wherein the Y adapter comprises a barcode in the double-stranded region.
21. The method of embodiment 20 wherein the Y adapter contains a random sequence of 1-10 nucleotide bases in the single strand region of each arm 22. The method of any of Embodiments 15 to 21, further comprising a step of removing proteins bound to the cfDNA, e.g., the cfDNA is treated with a protease to remove nucleosome or heterochromatin structures from the cfDNA fragments.
23. The method of any of Embodiments 15 to 22, wherein the method comprises generating a plurality of barcoded libraries with different barcodes.
24. The method of any of Embodiments 15 to 23, further comprising a step of purification.
25. The method of Embodiment 24, comprising a step of combining the plurality of barcoded libraries prior to the purification.
26. The method of any of Embodiments 15-25, wherein the spent medium is collected from a blastocyst culture of Day 5 to Day 7.
27. A method of identifying the genetic background of in-vitro fertilization (IVF) embryos comprising
   i) providing a sample containing cell-free DNA (cfDNA) from the spent medium in the culture of an IVF embryo;
   ii) repairing the cfDNA and preferably obtaining single base overhang fragments;
   iii) adding a directional barcoded adapter to each of the ends of the repaired cfDNA, thereby generating a barcoded library; and
   iv) determining the genome-wide heterozygous SNP profile or mitochondrial sequence by sequencing the barcoded library.
28. The method of Embodiment 27, wherein repairing the cfDNA comprises converting 5′ and/or 3′ overhang into blunt ends, and/or repairing the internal nicks.
29. The method of Embodiment 27, wherein the single base overhang fragments comprise a 3′ dA overhang to each end of the repaired fragments.
30. The method of Embodiment 27, wherein the barcoded library is sequenced by paired end sequencing.
31. The method of Embodiment 27, wherein the barcoded adapter is a Y adapter.
32. The method of Embodiment 31, wherein the Y adapter comprises a barcode in the double-stranded region.
33. The method of embodiment 32 wherein the Y adapter contains a random sequence of 1-10 bases in the single strand region of each arm 34. The method of any of Embodiments 27 to 33, further comprising a step of removing proteins bound to the cfDNA, e.g., the cfDNA is treated with a protease to remove nucleosome or heterochromatin structures from the cfDNA fragments.
35. The method of any of Embodiments 27 to 34, wherein the method comprises generating a plurality of barcoded libraries with different barcodes.
36. The method of any of Embodiments 27 to 34, further comprising a step of purification.
37. The method of Embodiment 36, comprising a step of combining the plurality of barcoded libraries prior to the purification.
38. The method of any of Embodiments 27-37, wherein the spent medium is collected from a blastocyst culture of Day 5 to Day 7.
39. An amplification-free method of determining the degree of non-embryonic DNA contamination in the spent medium in the culture of an IVF embryo, comprising
   i) generating a library by the method of any of Embodiments 15-34;
   ii) sequencing the library; and
   iii) calculating the mitochondrial DNA (mtDNA) to chromosome ratio, wherein a higher mtDNA to chromosome ratio is indicative of a lower degree of non-embryonic DNA contamination, and a lower mtDNA to chromosome ratio is indicative of a higher degree of non-embryonic DNA contamination, and optionally
   iv) assessing the X chromosome copy number when the embryo is a human 46,XY embryo.
40. The method of Embodiment 39, wherein the mtDNA to chromosome ratio is mtDNA to autosome ratio.
41. An amplification-free method of generating a library of cell-free DNAs (cfDNAs) from a biological material, the method comprising
   i. providing a sample containing cell-free DNA (cfDNA) from the biological material;
   ii. repairing the cfDNA and preferably obtaining single base overhang fragments; and
   iii. adding a barcoded adapter to each of the ends of the repaired cfDNA, thereby generating a barcoded library for sequencing.
42. The method of Embodiment 41, wherein repairing the cfDNA comprises converting 5′ and/or 3′ overhang into blunt ends, and/or repairing the internal nicks.
43. The method of Embodiment 41, wherein the single base overhang fragments comprise a 3′ dA overhang to each end of the repaired fragments.
44. The method of Embodiment 41, wherein the directional barcoded adapter is a Y adapter.
45. The method of Embodiment 44, wherein the Y adapter comprises a barcode in the double-stranded region.
46. The method of embodiment 44, wherein the Y adapter contains a random sequence of 1-10 bases in the single strand region of each arm 47. The method of any of Embodiments 41 to 46, further comprising a step of removing proteins bound to the cfDNA, e.g., the cfDNA is treated with a protease to remove nucleosome or heterochromatin structures from the cfDNA fragments.
48. The method of any of Embodiments 41-46, wherein the method comprises generating a plurality of barcoded libraries with different barcodes.
49. The method of any of Embodiments 41 to 47, further comprising a step of purification.
50. The method of Embodiment 48, comprising a step of combining the plurality of barcoded libraries prior to the purification.
51. A sequencing library generated by the method of any of Embodiments 16-26 and 41-50.
52. A method for the genetic testing of a sample, comprising steps of sequencing the library of Embodiment 51, and analyzing the data of the sequencing to determine the genome-wide heterozygous SNP profile or mitochondrial sequence.
53. An amplification-free method for preparing the sample for methylation profile analysis, the method comprising
   i) providing a sample containing cell-free DNA (cfDNA) from the biological material;
   ii) repairing the cfDNA and preferably obtaining single base overhang fragments; and
   iii) adding a directional barcoded adapter to each of the ends of the repaired cfDNA, thereby generating a barcoded library and avoiding PCR amplification which destroys the methylation patterns by converting methyl-C to C.
54. The method of Embodiment 53, wherein the adapter comprises unique Y adapters with incorporated barcoding.
55. The method of Embodiment 53 or 54, wherein barcoded libraries from different samples can be combined into a single sample in preparation.
56. The method of any of Embodiments 53 to 55, wherein the method comprises paired end sequencing of the Y adaptor library fragments to provide the full-length sequence of each fragment.
57. The method of embodiment any of Embodiments 53 to 56, wherein the Y adaptors can be modified to have a random sequence added to each primer strand in the single strand region.
58. The method of any of Embodiments 53 to 57, comprising enriching the methylated fraction of the combined cfDNA libraries, e.g., by specific capture of the methylated cfDNA fragments.
59. The method of Embodiment 58, wherein the specific capture is carried out using an antibody with the specificity for double or single stranded methylated DNA fragments, or using matrices that preferentially bind methylated DNA (mDNA) which can then be differentially eluted and sequenced.
60. The method of Embodiment 59, wherein the antibody is selected from anti-mC monoclonal or polyclonal antibodies or anti-ds antibodies from patients with SLE disease.
61. The method of Embodiment 59, wherein the antibody is bound to or captured by enrichment matrix such as beads to efficiently enrich the methylated fragments.
62. The method of any of Embodiments 58 to 61, wherein the captured methylated DNA fragments are released from the enrichment matrix and sequenced without further modifications.
63. The method of any of Embodiments 58 to 62, further comprising simply amplifying the enriched DNA fragments using primers homologous to the directional adapter sequences originally added prior to mDNA capture.
64. The method of any of Embodiments 53 to 63, further comprising the addition of protease, such as trypsin and proteinase K.
65. The method of Embodiment 64, wherein the protease is a heat labile proteinase K or membrane bound trypsin.
66. The method of any of Embodiments 53 to 65, wherein the methods further comprises sequencing the methylated DNA, e.g., by NGS.
67. The method of Embodiment 66, wherein the full DNA sequence of the methylated DNA fragments is obtained in NGS using the sequencing primers of the Y adaptors.
68. The method of Embodiment 66 or 67, wherein the NGS is performed in paired end NGS sequencing mode.
69. The method of any of Embodiments 53 to 65, wherein the methods further comprises sequencing the methylated DNA by nanopore sequencing, e.g., to obtain the full DNA sequence of long concatenated enriched methylated DNA fragments, in terms of the bases A, G, C, T and methylated C.
70. The method of any of Embodiments 53 to 69, wherein the method further comprises analysing the sequencing data derived from methylated DNA fragments.
71. The method of Embodiment 70, wherein the method comprises analysing the NGS data to map the methylated regions to chromosome bins and generate the methylation profiles for analysis.
72. The method of Embodiment 70, wherein the method comprises analysing the nanopore sequencing data to plot the genome-wide methylation sites for methylation analysis.
73. An amplification-free method for examining a selected genome region on a chromosome associated with a gene or a polymorphic site, the method comprising:
   i) providing a sample containing cell-free DNA (cfDNA);
   ii) repairing the cfDNA fraction and preferably obtaining single base overhang fragments;
   iii) adding a directional barcoded sequencing-compatible adapter to each of the ends of the repaired cfDNA, thereby generating a barcoded library;
   iv) enriching the library for the selected genome region; and
   v) sequencing the barcoded library.
74. The method of Embodiment 73, wherein the library is enriched by selectively binding any cfDNA homologous to the selected genome region with hybridization capture probes.
75. The method of Embodiment 73 or 74, wherein the sequence can be assessed for relative ratios of any polymorphic points identified in the captured DNA fragments.
76. The method of any of Embodiments 73 to 75, wherein the cfDNA can be cell associated but has been extracted and processed into a form compatible with the library preparation and subsequent manipulations.
77. The method of any of Embodiments 73 to 76, wherein the selected genome region can be sequenced and analysed for variations such as single base substitutions or small indels.
78. The method of any of claims 1 to 77, wherein the sample comprises cfDNA extracted from cells or tissues.
79. The method of any of claims 1 to 78, wherein the cfDNA is from tissue removed from an embryo by biopsy, and optionally, processed in a manner making it compatible with the subsequent library preparations.

### Advantages of the Invention

The present disclosure provides a method in which the sample preparation, such as library generation is simple, rapid, reliable and accurate. In the method of the invention, a whole genome amplification is not needed, at least, in the generation of library.

### EXAMPLES

### Example 1. Spent Embryo Medium Analysis

### 1.1. Samples

Spent medium after culture of blastocysts to day 5 or further, which was usually discarded at the completion of embryo development to the required blastocyst stage, was collected for the analysis.

### 1.2. Library Preparation

10 μL spent embryo culture media was mixed with 10 μl Repair Mix: {0.8 μL T4 DNA polymerase (Hunan Yearthbio) (3 U/μL), 0.4 μL rTaq (Hunan Yearthbio) (5 U/μL), 2 μL 10×T4 Ligase buffer (500 mM Tris-HCl, 100 mM MgCl₂, 10 mM ATP, 100 mM DTT), 1.5 μL 10 mM dNTPs) and 5.3 μL water} to give 20 μL in total. The mixture was incubated at 37° C. for 20 min, and then, at 72° C. for 30 min for end repair and “A addition”.

The product was then mixed with 10 μl Adapter Mix: {0.6 μL ADT-FL (1 μM Hunan Yearthbio), 1 μL T4 Ligase (600 U/μL), 1 μL 10×T4 Ligase buffer (500 mM Tris-HCl, 100 mM MgCl₂, 10 mM ATP, 100 mM DTT), PEG6000 (Sigma) (final concentration of 25-27%) water to 10 μl} total volume ˜30 μl. The barcode Y adapter ligation mix was incubated at 20° C. for 15 min, to obtain the barcoded library and then heated at 65° C. for 10 min to inactivate the ligase.

Libraries with different barcodes are then combined and the modified cfDNA libraries are purified using 0.8X Agencourt AMPure beads according to the manufacturer's directions. After washing with 80% fresh ethanol to remove unused reagents and Y adapters, the modified cfDNA was eluted from the beads using 20-40 μl DB pH 8.

### 1.3. Sequencing

The combination of the barcoded libraries were sequenced on a NGS platform (Illumina NovaSeq, Applied Biosystems S5, BGI MGI-T7).

For each library to get relative sequencing reads, libraries were pooled and purified. The pooled libraries were adjusted to 10 pmole per μL in resuspension buffer (RSB) and injected into the Illumina NovaSeq flow cell. Sequencing was run in the 2×150 paired end mode.

### 1.4. Read Processing

The reads were processed with Redis (for data storage), Burrow-Wheeler-Aligner (for mapping), fastq count (for raw data QC), GCcorrect (for GC correction), CNVcalling.R (for the calculation of profiles) or derivatives thereof.

### Example 2. The Size Analysis of the Native cfDNA in Embryo Spent Media

The size of the native cfDNA in embryo spent media was analyzed by paired end sequencing of the libraries followed by mapping against the reference genome and fragment size estimation

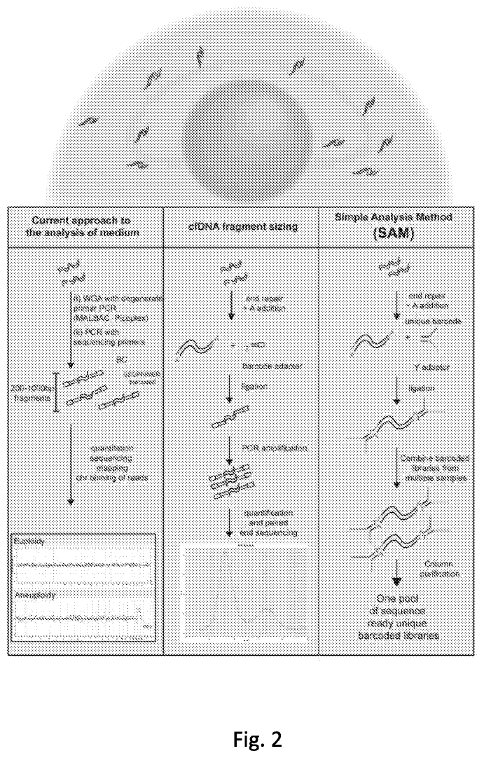

As shown in FIG. 2, a major peak of ˜170 bp fragments was consistently seen in all 4 cfDNA samples from embryo culture media, while minor apparent multimer peaks of ˜340 bp and ˜510 bp were also seen.

It should be noted that the major population of cfDNA molecules, which is ˜170 bp or less in length, is not readily amplifiable by current PCR based WGA methods.

### Example 3. Copy Number Analysis

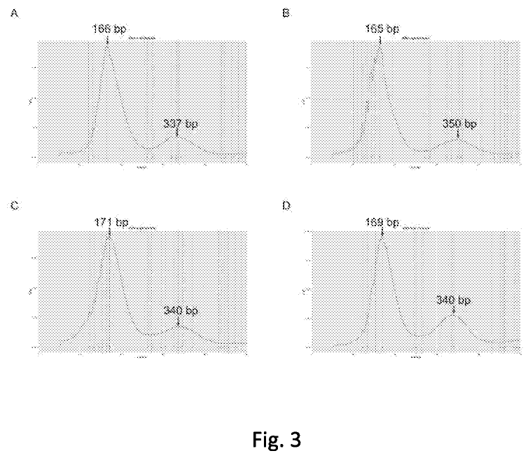

Pair end sequencing reads were sized after alignment to the reference genome and split into two size fractions of <200 bp and >200 bp. The reads were mapped to human genome, and results were shown in FIG. 3 with Chromosome 21, Chromosome 22 and Chromosome X as examples (a 46, XX sample). The fragments from the p arm of chromosome 21 and 22 cannot be mapped because it is only a repetitive DNA structure.

Copy number results were similar from the two size fractions.

### Example 4. Determination of Sex Chromosome Balance

The sex chromosome balance was determined by the copy number of the X chromosome.

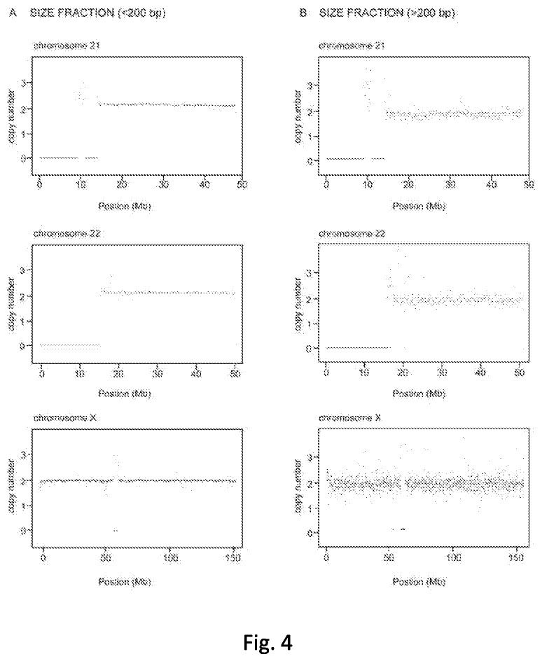

FIG. 4A showed the identification of a 46, XY sample indicating that the embryo comprises two copies of each autosome, and one copy of Chromosome X.

FIG. 4B showed the identification of a 46, XX sample indicating that the embryo comprises two copies of each autosome and Chromosome X.

FIG. 4C showed the identification of Y chromosome-specific sequences from a 46, XY and a 46, XX sample, indicating that the reads corresponding to Chromosome Y is present in the data from the 46, XY sample, but is absent in the data from the 46, XX sample. The figure showed fragments that only plot to the Y chromosome. Similarly, other fragments plotted against the other autosomes are specific to that chromosome. Sequences that are shared across chromosomes are removed during the initial analysis since they cannot be assigned to a single location).

### Example 5. Detection of Embryo Euploidy and Aneuploidy

The sequencing data were analyzed for detecting the embryo euploidy or aneuploidy. Since chromosomes exist as one copy, 2 copies or more and in the case of a female 0 copies of Y. Since autosomes are by convention copy 2, the sex chromosomes can be compared to an autosome and will give a relative copy number of 1 or 2 (0 for Y in females).

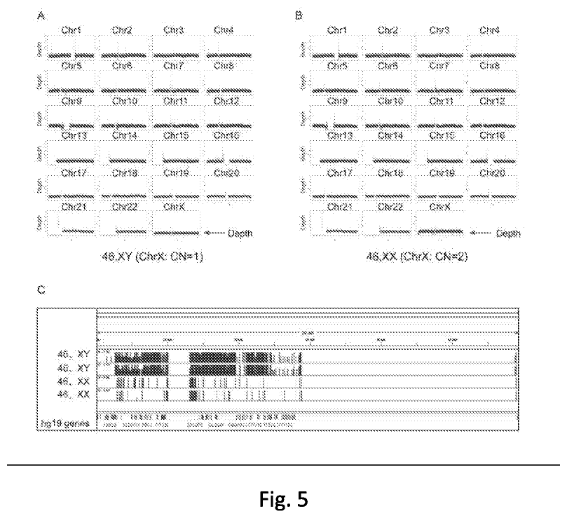

FIG. 5 showed the 24-chromosome plots. FIGS. 5A and 5B showed the results from a euploid embryo, 46, XY and a euploid embryo, respectively, while FIG. 5C showed result from an ancuploid embryo, in which the reads corresponding to Chromosome 15 showed a copy number of one, indicating that only one Chromosome 15 is present in the embryo.

### Example 6. Predicting Non-Embryonic DNA Contamination by Calculating mtDNA: Autosome Ratios

The mtDNA level (total 48 million base pairs or less) is typically only 1% or even less of the cell DNA content. Comparisons of a big number to a very small number can end up insensitive. Since each chromosome is a different size, ranging from 250 million base pairs for the biggest one to around 60 million base pairs for the smallest one, it can be more sensitive to compare mtDNA to a chromosome more relative in DNA content. It need not be a single chromosome since multiple independent comparisons may achieve more stable estimates.

Therefore, relative mtDNA reads were compared to autosomal reads (either total or selected chromosomes) to predict the non-embryonic DNA contamination.

### Example 7. Analysis of Mitochondrial DNA

The mitochondrial DNA data from the maternal parent(s) were used as reference.

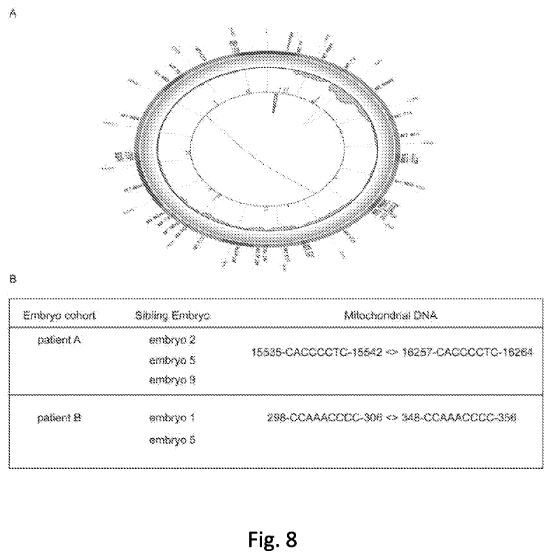

FIG. 8A showed the mapping and read coverage of mitochondrial DNA sequences. mtDNA reads were aligned against the mtDNA reference genome. The number of reads at any location is indicative of the depth and copy number of mtDNA relative to autosomal fragments.

FIG. 8B. showed the discrimination of embryos in different patient cohorts by mitochondrial DNA polymorphisms In particular, among one population of embryos, embryo 2, embryo 5 and embryo 9 showed the same polymorphism in the mitochondrial DNA reads with patient A, and thus, were determined to belong to the same cohort. Among another population of embryos, embryo 1, and embryo 5 showed the same polymorphism in the mitochondrial DNA reads with patient B, and thus, were determined to belong to the same cohort.

### Example 8. Enrichment of Methylated DNA Fragments

### 8.1 Samples

Samples included human genomic DNA isolated from the isolated cell fraction of blood and cfDNA.

The genomic DNA was isolated and purified according to standard protocols. A portion of the DNA was sonicated to produce an average fragment size of ˜200 bp. Sources included individuals with known malignancies and individuals without any known underlying diseases.

cfDNA was prepared from the serum/plasma fraction of blood from patients according to standard protocols. Patients included those with known bowel cancer and those with no known malignancies.

### 8.2 Library Preparation

160 ng sheared genomic DNA or purified cfDNA from 1 ml of plasma (˜10-20 ng) was used to prepare a library as follows:

10 μL spent embryo culture media was mixed with 10 μl Repair Mix: {0.8 μL T4 DNA polymerase (Hunan Yearthbio) (3 U/μL), 0.4 μL rTaq (Hunan Yearthbio) (5 U/μL), 2 μL 10×T4 Ligase buffer (500 mM Tris-HCl, 100 mM MgCl₂, 10 mM ATP, 100 mM DTT), 1.5 μL 10 mM dNTPs) and 5.3 μL water} to give 20 μL in total. The mixture was incubated at 37° C. for 20 min, and then, at 72° C. for 30 min for end repair and “A addition”.

The product was then mixed with 10 μl Adapter Mix: {0.6 μL ADT-FL (1 μM Hunan Yearthbio), 1 μL T4 Ligase (600 U/μL), 1 μL 10×T4 Ligase buffer (500 mM Tris-HCl, 100 mM MgCl₂, 10 mM ATP, 100 mM DTT), PEG6000 (Sigma) (final concentration of 25-27%) water to 10 μl} total volume ˜30 μl. The barcode Y adapter ligation mix was incubated at 20° C. for 15 min, to obtain the barcoded library and then heated at 65° C. for 10 min to inactivate the ligase.

### 8.3 Methyl DNA Enrichment

A Methylated-DNA IP Kit (Zymo Research, catalog No D5101) was used according to manufacturer's instructions to prepare the enriched mDNA fraction.

Essentially: The library prepared fragments from 8.2 was combined with DNA denaturing buffer to give a total volume of 50 μl. This was heated at 98° C. for 5 minutes. Sequentially, 250 μl MIP Buffer was prepared by addition of 15 μl ZymoMag Protein A and 0.8 μl Anti-Methylcytosine antibody. The denatured DNA was added and the mix incubated at 37° C. for 0.5-1 hour. Beads were separated on a magnetic rack and the supernatant was discarded. Beads were then washed two times with 500 μl MIP buffer. DNA was eluted with 500 μl DNA Elution Buffer. Tubes were then incubated at 75° C. for 5 minutes followed by a 2 minute spin in a microcentrifuge. Enriched DNA is in the supernatant fraction.

### 8.4. Sequencing

The combination of the barcoded libraries were sequenced on a NGS platform (Illumina NovaSeq, Applied Biosystems S5, BGI MGI-T7).

### 8.5. Read Processing

The reads were processed with Redis (for data storage), Burrow-Wheeler-Aligner (for mapping), fastq count (for raw data QC), GCcorrect (for GC correction), CNVcalling.R (for the calculation of profiles) or derivatives thereof.

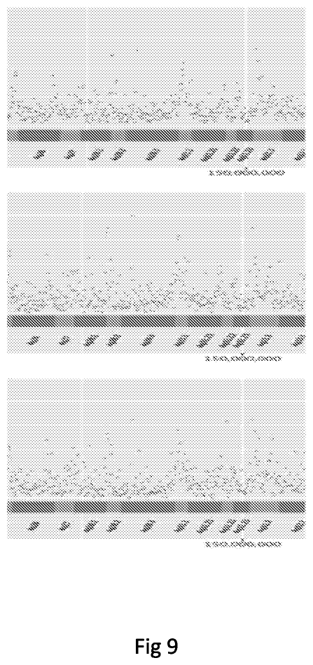

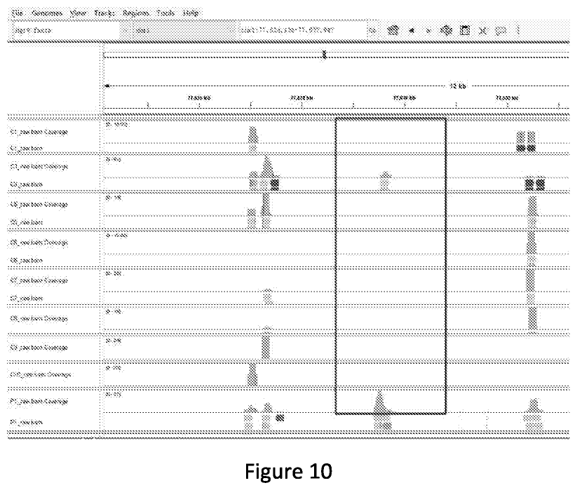

The results were shown in FIGS. 9 and 10. In particular, FIG. 9 showed a randomly selected region of chromosome 4 compared for captured fragments across three different samples. This demonstrated overall reliability of the process across samples. FIG. 10 showed regions of both similarity and difference in mDNA profiles from different samples demonstrating the usefulness of the approach in examining methylation commonalities and differences between samples.

### Example 9 Detection and Quantification of Minor Alleles

### 9.1 Samples

Samples included:
   plasma from women pregnant with a known male fetus, and plasma from a female. Plasma from a male
   cfDNA was prepared from the serum/plasma fraction of blood according to standard protocols.

### 9.2 Library Preparation

cfDNA was purified from 1 ml of plasma (˜10-20 ng) and was used to prepare a library as follows:

10 μL DNA was mixed with 0.8 μL T4 DNA polymerase (3 U/μL), 0.4 μL rTaq (Hunan Yearthbio) (5 U/μL), 3.5 μL Buffer 1 (2 μL 10×T4 Ligase buffer (500 mM Tris-HCl, 100 mM MgCl₂, 10 mM ATP, 100 mM DTT) and 1.5 μL 10 mM dNTPs) and 5.3 μL water (20 UL in total). The mixture was incubated at 37° C. for 20 min, and then, at 72° C. for 30 min for end repair and “A addition”.

The product was then mixed with 0.6 μL ADT-FL (1 μM), 1 μL T4 Ligase (600 U/μL), 8.7 μL Buffer 2 (1 μL 10×T4 Ligase buffer (500 mM Tris-HCl, 100 mM MgCl₂, 10 mM ATP, 100 mM DTT), PEG6000 (Sigma) (to give a final concentration in the ligation mix of 6%, adding water to 8.7 μL). The mixture was incubated at 20° C. for 15 min, for ligating a barcoded Y adapter, thereby obtaining a barcoded library and then incubated at 65° C. for 10 min to deactivate the ligase enzyme.

### 9.3 Polymorphic SNP Enrichment

SNP enrichment was performed using the cfDNA Library NanoID Panel Capture Kit according to the manufacturer's directions (DeepL).

Briefly, the purified cfDNA library was dried down after the addition of Sul of human Cot DNA and 2 μl NadPrep NanoBlockers. The dried down probe was redissolved in hybridization mix containing 8.5 μl Hyb #1, 2.7 μl Hyb #2 and 6 μl NanoID Panel Probe. After denaturing at 95° C. for 30 seconds the mix was allowed to hybridize overnight at 65° C. Streptavidin beads (50 μl) were prepared according to the manufacturer's instructions and added to the hybridization mix. This was kept at 65° C. for 45 minutes with intermediate mixing every 10-12 minutes. 100 μl of Wash Buffer 1 is added to the library/bead mix and the beads are separated on a magnetic rack. The beads are washed 2 times in Wash Buffer, 65° C.×5 minutes, one time in Wash Buffer 1 (2 minutes at room temperature), once in Wash Buffer 2 (2 minutes at room temperature), once in Wash Buffer 3 (2 minutes at room temperature) and resuspended in 23 μl H₂O. The bead mix is added to a PCR reaction mix comprising 25 μl HiFi HotStart Ready Mix, 2 μL P5+P7 Primer mix (25 μM) and cycled 1×98° C.×45 seconds; 15×(98° C.×15 seconds 60° C.×30 seconds/72° C.×30 seconds); 1×72° C.×1 minute. The library was then purified using 54 μl VAHTS DNA Clean Beads according to manufacturer's instructions.

### 9.4. Sequencing

The combination of the barcoded libraries were sequenced on a NGS platform (Illumina NovaSeq, Applied Biosystems S5, BGI MGI-T7).

### 9.5. Read Processing

The reads were processed with Redis (for data storage), Burrow-Wheeler-Aligner (for mapping), fastq count (for raw data QC), GCcorrect (for GC correction), CNVcalling.R (for the calculation of profiles) or derivatives thereof.

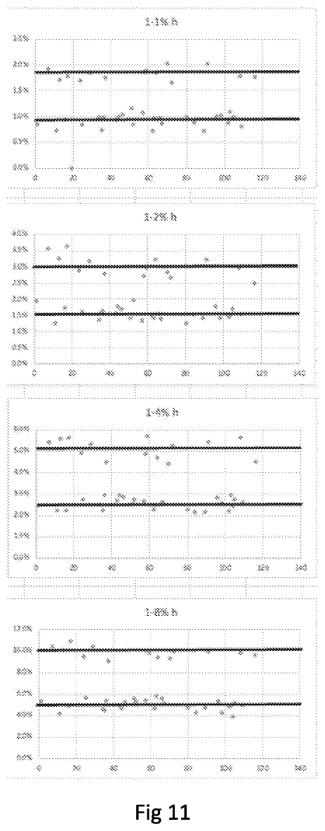

The results were shown in FIG. 11. In particular, FIG. 11 showed the SNP profiles for minor alleles in mixed genetic samples demonstrating utility in transplant monitoring, pregnancy monitoring or any other application such as mixed biological samples where different genetic contributions are present and relative amount estimates may be a useful analysis.

1. An amplification-free method for analyzing cell-free DNA (cfDNA) in a biological sample comprising
i) providing a sample containing cell-free DNA (cfDNA);
ii) repairing the cfDNA fraction to obtain single base overhang fragments;
iii) adding a barcoded adapter to each of the ends of the repaired cfDNA, thereby generating a barcoded library for sequencing; and
iv) sequencing the barcoded library or prior to sequencing, processing through further enrichment steps.

2. An amplification-free method for the preimplantation genetic testing (PGT) of in-vitro fertilization (IVF) embryos comprising
i) providing a sample containing cell-free DNA (cfDNA) from spent medium in the culture of an IVF embryo;
ii) repairing the cfDNA fraction to obtain blunt ended fragments;
iii) adding a barcoded adapter to each of the ends of the repaired cfDNA, thereby generating a barcoded library for sequencing; and
iv) sequencing the barcoded library.

3. The method of claim 1 or 2, wherein repairing the cfDNA comprises converting 5′ and/or 3′ overhang into blunt ends, and/or repairing the internal nicks.

4. The method of claim 1 or 2, wherein the single base overhang fragments comprise a 3′ dA overhang to each end of the repaired fragments.

5. The method of claim 1 or 2, wherein the barcoded library is sequenced by paired end sequencing.

6. The method of claim 1 or 2, wherein the barcoded adapter is an Y adapter.

7. The method of claim 6, wherein the Y adapter comprises a barcode in the double-stranded region.

8. The method of claim 6, wherein the Y adapter comprises a random nucleotide sequence of 1-10 bases in each arm of the single-stranded region.

9. The method of any of claims 1 to 8, further comprising a step of removing proteins bound to the cfDNA.

10. The method of any of claims 1 to 9, wherein the method comprises generating a plurality of barcoded libraries with different barcodes.

11. The method of any of claims 1 to 10, further comprising a step of purification.

12. The method of claim 11, comprising a step of combining the plurality of barcoded libraries prior to the purification.

13. The method of any of claims 1 to 12, further comprising the analysis of the sequencing data to obtain a 24-chromosome profile for determining chromosome ploidy status of an embryo.

14. The method of any of claims 1 to 12, further comprising the analysis of the sequencing data to obtain a chromosome X and Y profile for determining sex chromosome balances.

15. The method of claim 14, further comprising calculating the copy number of X chromosome by comparison to autosomal regions.

16. The method of any of claims 1-15, wherein the spent medium is collected from a blastocyst culture of Day 5 to Day 7.

17. An amplification-free method of generating a library for the preimplantation genetic testing (PGT) of in-vitro fertilization (IVF) embryos comprising
i) providing a sample containing cell-free DNA (cfDNA) from the spent medium in the culture of an IVF embryo;
ii) repairing the cfDNA to obtain blunt ended fragments; and
iii) adding a barcoded adapter to each of the ends of the repaired cfDNA, thereby generating a barcoded library for sequencing.

18. The method of claim 17, wherein repairing the cfDNA comprises converting 5′ and/or 3′ overhang into blunt ends, and/or repairing the internal nicks.

19. The method of claim 15, wherein the single base overhang fragments comprise a 3′ dA overhang to each end of the repaired fragments.

20. The method of claim 15, wherein the barcoded adapter is an Y adapter.

21. The method of claim 18, wherein the Y adapter comprises a barcode in the double-stranded region.

22. The method of claim 21, wherein the Y adapter comprises a random nucleotide sequence of 1-10 bases in each arm of the single-stranded region.

23. The method of any of claims 17 to 22, further comprising a step of removing proteins bound to the cfDNA.

24. The method of any of claims 17 to 23, wherein the method comprises generating a plurality of barcoded libraries with different barcodes.

25. The method of any of claims 17 to 24, further comprising a step of purification.

26. The method of claim 25, comprising a step of combining the plurality of barcoded libraries prior to the purification.

27. The method of any of claims 17-26, wherein the spent medium is collected from a blastocyst culture of Day 5 to Day 7.

28. A method of identifying the genetic background of in-vitro fertilization (IVF) embryos comprising
i) providing a sample containing cell-free DNA (cfDNA) from the spent medium in the culture of an IVF embryo;
ii) repairing the cfDNA to obtain blunt ended fragments; and
iii) adding a barcoded adapter to each of the ends of the repaired cfDNA, thereby generating a barcoded library; and
iv) determining the genome-wide heterozygous SNP profile or mitochondrial sequence by sequencing the barcoded library.

29. The method of claim 28, wherein repairing the cfDNA comprises converting 5′ and/or 3′ overhang into blunt ends, and/or repairing the internal nicks.

30. The method of claim 28, wherein the single base overhang fragments comprise a 3′ dA overhang to each end of the repaired fragments.

31. The method of claim 28, wherein the barcoded library is sequenced by paired end sequencing.

32. The method of claim 28, wherein the barcoded adapter is an Y adapter.

33. The method of claim 32, wherein the Y adapter comprises a barcode in the double-stranded region.

34. The method of claim 33, wherein the Y adapter comprises a random nucleotide sequence of 1-10 bases in each arm of the single-stranded region.

35. The method of any of claims 28 to 34, further comprising a step of removing proteins bound to the cfDNA.

36. The method of any of claims 28 to 34, wherein the method comprises generating a plurality of barcoded libraries with different barcodes.

37. The method of any of claims 28 to 36, further comprising a step of purification.

38. The method of claim 36, comprising a step of combining the plurality of barcoded libraries prior to the purification.

39. The method of any of claims 28-38, wherein the spent medium is collected from a blastocyst culture of Day 5 to Day 7.

40. An amplification-free method of determining the degree of non-embryonic DNA contamination in the spent medium in the culture of an IVF embryo, comprising
i) generating a library by the method of any of claims 17-27;
ii) sequencing the library; and
iii) calculating the mitochondrial DNA (mtDNA) to chromosome ratio, wherein a higher mtDNA to chromosome ratio is indicative of a lower degree of non-embryonic DNA contamination, and a lower mtDNA to chromosome ratio is indicative of a higher degree of non-embryonic DNA contamination.

41. The method of claim 40, wherein the mtDNA to chromosome ratio is mtDNA to autosome ratio.

42. An amplification-free method of generating a library of cell-free DNAs (cfDNAs) from a biological material, the method comprising
i) providing a sample containing cell-free DNA (cfDNA) from the biological material;
ii) repairing the cfDNA to obtain blunt ended fragments; and
iii) adding a barcoded adapter to each of the ends of the repaired cfDNA, thereby generating a barcoded library for sequencing.

43. The method of claim 42, wherein repairing the cfDNA comprises converting 5′ and/or 3′ overhang into blunt ends, and/or repairing the internal nicks.

44. The method of claim 42, wherein the single base overhang fragments comprise a 3′ dA overhang to each end of the repaired fragments.

45. The method of claim 42, wherein the barcoded adapter is an Y adapter.

46. The method of claim 42, wherein the Y adapter comprises a barcode in the double-stranded region.

47. The method of claim 42, wherein the Y adapter comprises a random nucleotide sequence of 1-10 bases in each arm of the single-stranded region.

48. The method of any of claims 42 to 47, further comprising a step of removing proteins bound to the cfDNA.

49. The method of any of claims 42 to 48, wherein the method comprises generating a plurality of barcoded libraries with different barcodes.

50. The method of any of claims 42 to 49, further comprising a step of purification.

51. The method of claim 50, comprising a step of combining the plurality of barcoded libraries prior to the purification.

52. An amplification-free method for preparing the sample for methylation profile analysis, the method comprising
i) providing a sample containing cell-free DNA (cfDNA) from the biological material;
ii) repairing the cfDNA to obtain single base overhang fragments; and
iii) adding a directional barcoded adapter to each of the ends of the repaired cfDNA, thereby generating a barcoded library and avoiding PCR amplification which destroys the methylation patterns by converting methyl-C to C.

53. The method of claim 52, wherein the library comprises both methylated and non-methylated cfDNA.

54. The method of claim 52 or 53, further comprising the enrichment of the methylated fraction of the library.

55. The method of any of claims 1 to 54, wherein the sample comprises cfDNA extracted from cells or tissues.

56. The method of any of claims 1 to 54, wherein the cfDNA is from tissue removed from an embryo by biopsy.
